SEQUENCE LISTING (3 sequences)

SEQ ID NO: 1 - DNA, 43 bp, Artificial Sequence
  Synthetic
ttagatggttttcctgggccgcgggacactccgcctttttttt

SEQ ID NO: 2 - RNA, 22 nt, Homo sapiens
uaacacugucugguaaagaugg

SEQ ID NO: 3 - DNA, 48 bp, Artificial Sequence
  Synthetic
  misc_feature: N= at least one T
  misc_feature (43)..(43): n is a, c, g, or t
cagacagtgttatttggcggagtgtcccgcggcccaggaccantcttt

(19) United States
(12) Patent Application Publication
(10) Pub. No.: US 2021/0318296 A1
(43) Pub. Date: Oct. 14, 2021

(54) INTRAMOLECULAR KINETIC PROBES

(71) Applicant: The Regents of the University of Michigan, Ann Arbor, MI (US)

(72) Inventors: Alexander Johnson-Buck, Ann Arbor, MI (US); Muneesh Tewari, Ann Arbor, MI (US); Nils G. Walter, Ann Arbor, MI (US)

(21) Appl. No.: 17/259,601

(22) PCT Filed: Jul. 23, 2019
(86) PCT No.: PCT/US2019/043022

§ 371 Date: Jan. 12, 2021

Related U.S. Application Data
(60) Provisional application No. 62/702,670, filed on Jul. 24, 2018.

(51) Int. Cl. G01N 33/542; G01N 33/557; G01N 33/53; G01N 21/64

(52) U.S. Cl. CPC G01N 33/542; G01N 33/557; G01N 21/6486; G01N 33/5308; G01N 33/5302

(57) ABSTRACT

Provided herein is technology relating to detecting analytes and particularly, but not exclusively, to methods, compositions and systems for detecting, characterizing, identifying, and/or quantifying analytes.

This application claims priority to U.S. provisional patent application Ser. No. 62/702,670, filed Jul. 24, 2018, which is incorporated herein by reference in its entirety.

### STATEMENT REGARDING FEDERALLY SPONSORED RESEARCH OR DEVELOPMENT

This invention was made with government support under W911NF-12-1-0420 awarded by the U.S. Army/Army Research Office, and GM062357, CA204560, and CA229023 awarded by the National Institutes of Health. The government has certain rights in the invention.

### FIELD

Provided herein is technology relating to detecting analytes and particularly, but not exclusively, to methods, compositions, and systems for detecting, characterizing, identifying, and/or quantifying analytes.

### BACKGROUND

The sensitive and specific detection of low-abundance analytes (e.g., including, but not limited to proteins, nucleic acids, lipids, and small molecules (e.g., drugs, metabolites, cofactors, etc.)) plays an important role in in vitro diagnostics as well as in biomedical research. Such detection is generally carried out using molecular reagents (e.g., antibodies, hybridization probes) that have high affinity and specificity for the analyte of interest, and that produce a detectable signal only in the presence of the target analyte. However, affinity probes have a finite specificity and affinity for the target analyte (see, e.g., Zhang et al. (2012) “Optimizing the specificity of nucleic acid hybridization. Nat. Chem. 4, 208-214), thus making it challenging to detect extremely low concentrations of analyte with high confidence. This is a significant problem because biomarkers of interest are often present at vanishingly small concentrations (e.g., at sub-femtomolar concentrations: see, e.g., Lee et al. (2001) “Quantitation of genomic DNA in plasma and serum samples: higher concentrations of genomic DNA found in serum than in plasma.” Transfusion (Paris) 41, 276-282; Mitchell et al. (2008) “Circulating microRNAs as stable blood-based markers for cancer detection” Proc. Natl. Acad. Sci. U.S.A. 105, 10513-18; Bettegowda et al. (2014) “Detection of Circulating Tumor DNA in Early- and Late-Stage Human Malignancies”Sci. Transl. Med. 6, 224ra24-224ra24; Husain et al. (2017) “Monitoring Daily Dynamics of Early Tumor Response to Targeted Therapy by Detecting Circulating Tumor DNA in Urine” Clin. Cancer Res. 23, 4716-23).

While sensitivity can be increased by amplifying the signal or producing many copies of the analyte, binding of the probe reagents to spurious analytes (such as partly homologous amino acid or peptide sequences) or to imaging surfaces generally imposes a finite limit of detection at approximately thousands of molecular copies or more (see, e.g., Cohen (2017) “Digital direct detection of microRNAs using single molecule arrays” Nucleic Acids Res. 45, e137-e137).

### SUMMARY

Accordingly, provided herein is a technology relating to detecting analytes and particularly, but not exclusively, to methods, compositions, and systems for detecting, characterizing, identifying, and/or quantifying analytes. In some embodiments, the technology detects, characterizes, identifies, and/or quantifies analytes present in low abundance and/or at low concentration (e.g., sub-femtomolar concentration). In some embodiments, the technology detects, characterizes, identifies, and/or quantifies analytes with single-molecule resolution.

A method for detecting single molecules of analytes with nearly arbitrarily high specificity using kinetic fingerprinting has been described previously (see, e.g., Johnson-Buck et al. (2015) “Kinetic fingerprinting to identify and count single nucleic acids. Nat. Biotechnol. 33, 730-32; Walter et al. “Detection of nucleic acids”, U.S. Pat. App. Ser. No. 14/589,467, each of which is incorporated herein by reference in its entirety; see also Int'l Pat. App. No. PCT/US2017/016977, incorporated herein by reference in its entirety).

These extant methods comprise, in some embodiments, use of a query probe in trans, which impairs fast probe association/dissociation kinetics for some analytes and, consequently, limits the speed of data acquisition in some experiments. The present disclosure relates to a technology comprising use of a dynamic nanomachine that undergoes conformational changes with characteristic kinetics in the presence of a specific molecular analyte. Accordingly, the technology comprises observing, recording, and analyzing these conformational changes to determine the presence or absence of the analyte with high confidence. Some embodiments further relate to characterizing, identifying, and/or quantifying analytes.

For example, in some embodiments, the technology provides a biosensor that incorporates several components into a discrete, covalently or non-covalently bonded nanostructure, e.g:

A capture probe moiety (C) that binds to one epitope, moiety, or region of the target analyte (T) with high thermodynamic stability (e.g., a melting temperature more than 10 degrees C. above the measurement temperature, or that associates with T for an average lifetime more than 10 times longer than the observation period).

A query probe moiety (Q) that binds to a different epitope, moiety, or region of T with moderate to low thermodynamic stability (e.g., a melting temperature within 10 degrees C. of the measurement temperature, or that associates with T for an average lifetime no more than ⅕th as long as the observation period).

Labels L1 and L2 associated with Q and C, respectively, whose mutual distance is measurably smaller when both Q and C are associated with T than when only C is bound by T. For instance, in some embodiments L1 and L2 comprise a Forster resonance energy transfer (FRET) pair of a donor fluorophore and an acceptor fluorophore. In some embodiments, L1 and L2 comprise a fluorophore and a quencher.

Thus, in some embodiments when target T binds to the biosensor, T binds stably to C (e.g., on a thermodynamic stable time scale) but only transiently to Q (e.g., on a kinetically transient time scale), and the biosensor will undergo measurable transitions between a State 1 in which both Q and C are bound to T, and a second State 2 in which only C is bound to T (See, e.g., FIG. 1).

In some embodiments, the biosensor incorporates one or more of the following optional components:

A decoy moiety (D) that binds weakly to Q, thus competing with T for binding of Q and prolonging the lifetime of State 2 relative to State 1. In some embodiments, D is connected to the biosensor covalently or non-covalently at all times during the measurement (See, e.g., FIG. 1a). In some embodiments, D is provided in trans (e.g., in solution) such that it is only bound to the biosensor when it is bound to Q (See, e.g., FIG. 2c).

An anchor moiety (A) for immobilization to a surface or other phase boundary for easy measurement. For example, in some embodiments A is, e.g., biotin, streptavidin, digoxigenin, anti-digoxigenin, an azide or alkyne moiety for click chemistry-mediated immobilization, or another affinity tag or functional group capable of covalent or non-covalent interaction with a surface or phase boundary.

During the development of embodiments of the technology described herein, experiments were conducted in which the biosensor comprised a set of designed synthetic nucleic acid sequences and incorporated both a decoy sequence and a biotin anchor (see, e.g., FIG. 3a). Data were collected during these experiments using a single-molecule FRET microscopy technique (FIG. 3b).

The technology provides advantages relative to other extant analyte detection technologies. For example, the present technology detects single analyte molecules with high confidence, which is not generally achievable for non-nucleic acid analytes detected with other technologies. Further, the technology does not require amplifying target analytes (e.g., by nucleic acid amplification technologies such as polymerase chain reaction (PCR), NASBA, RPA, Invader, etc.). In some embodiments, the technology provides alternate, improved acquisition times and is more amenable to multiplexed detection of multiple analytes than similar technologies (see, e.g., Johnson-Buck et al. (2015) “Kinetic fingerprinting to identify and count single nucleic acids. Nat. Biotechnol 33, 730-32; Walter et al. “Detection of nucleic acids”, U.S. patent application Ser. No. 14/589,467, each of which is incorporated herein by reference in its entirety; see also Int'l Pat. App. No. PCT/US2017/016977, incorporated herein by reference in its entirety). Further, in some embodiments the technology provides a much lower detection limit than other technologies based on ELISA, microarray, or the nCounter system.

In some embodiments, the technology described herein provides a biosensor. In some embodiments, the biosensor comprises a capture probe moiety and a query probe moiety. In some embodiments, the capture probe moiety comprises a first label and the query probe moiety comprises a second label. In some embodiments, the capture probe binds to a target analyte with a high thermodynamic stability and the query probe moiety binds transiently to the target analyte. In some embodiments, the first label and the second label are a FRET pair. In some embodiments, the first label and the second label are a fluor-quencher pair. In some embodiments, the capture probe moiety binds to the target analyte to form a complex comprising the capture probe moiety and the target analyte. In some embodiments, the complex has a melting temperature more than 10° C. above the measurement temperature of the assay. In some embodiments, the capture probe moiety binds to said target analyte to form a complex comprising the capture probe moiety and the target analyte. In some embodiments, the complex has an average lifetime that is more than 5 times as long as the observation period.

In some embodiments, the observation period is a time interval that, is tens to hundreds to thousands of seconds, e.g., 5, 10, 15, 20, 25, 30, 35, 40, 45, 50, 55, or 60 seconds; e.g., 5, 10, 15, 20, 25, 30, 35, 40, 45, 50, 55, or 60 minutes; e.g., 1, 1.5, 2, 2.5, or 3 hours). In some embodiments, the acquisition time is approximately 1 to 10 seconds to 1 to 10 minutes (e.g., approximately 1 to 100 seconds, e.g., 1, 2, 3, 4, 5, 6, 7, 8, 9, 10, 11, 12, 13, 14, 15, 16, 17, 18, 19, 20, 30, 40, 50, 60, 70, 80, 90, or 100 seconds, e.g., 1 to 100 minutes, e.g., 1, 2, 3, 4, 5, 6, 7, 8, 9, 10, 11, 12, 13, 14, 15, 16, 17, 18, 19, 20, 30, 40, 50, 60, 70, 80, 90, or 100 minutes. In some embodiments, the acquisition time is 0.1-20.0 seconds (e.g., 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, or 20.0 seconds).

In some embodiments, the query probe moiety transiently binds to the target analyte to form a complex comprising the capture query probe moiety and the target analyte. In some embodiments, the complex has a melting temperature within 10° C. of the measurement temperature of the assay. In some embodiments, the query probe moiety transiently binds to the target analyte to form a complex comprising the capture query probe moiety and the target analyte. In some embodiments, the complex has an average lifetime that no more than ⅕ as long as the observation period. In some embodiments, the capture probe moiety and the query probe moiety are covalently linked. In some embodiments, the biosensor comprises a covalently attached anchor moiety. In some embodiments, the biosensor further comprises a decoy moiety. In some embodiments, the decoy moiety transiently binds to the target analyte to form a complex comprising the capture query probe moiety and the target analyte. In some embodiments, the complex has a melting temperature within 10° C. of the measurement temperature of the assay. In some embodiments, the decoy moiety transiently binds to the target analyte to form a complex comprising the capture query probe moiety and the target analyte. In some embodiments, the complex has an average lifetime that is no more than ⅕ as long as the observation period.

In some embodiments, the decoy moiety comprises a label or quencher. In some embodiments, the capture probe moiety comprises an antibody. In some embodiments, the query probe moiety comprises an antibody. In some embodiments, the capture probe moiety comprises a nucleic acid. In some embodiments, the query probe moiety comprises a nucleic acid.

In some embodiments, the technology provides methods. In some embodiments, methods comprise contacting an analyte with a biosensor comprising a capture probe moiety and a query probe moiety, wherein said capture probe moiety comprises a first label and said query probe moiety comprises a second label; and recording a fluorescence signal indicating the association and dissociation of the query probe with the analyte as a function of time to produce fluorescence transition data. In some embodiments, methods comprise forming a thermodynamically stable complex between said capture probe and said analyte. In some embodiments, methods comprise repeatedly forming a transient complex between said query probe and said analyte. In some embodiments, methods comprise counting fluorescence transitions in said fluorescence transition data. In some embodiments, the biosensor comprises an anchor moiety and the method comprises immobilizing said biosensor to a surface. In some embodiments, methods comprise providing or obtaining a sample. In some embodiments, methods comprise providing a decoy moiety. In some embodiments, methods comprise contacting said query probe with a decoy moiety. In some embodiments, methods comprise providing an excitation wavelength. In some embodiments, methods comprise detecting an emission wavelength. In some embodiments, methods comprise recording an emission wavelength as a function of time. In some embodiments, methods comprise recording a time-dependent signal of an emission wavelength. In some embodiments, methods comprise monitoring fluorescence at a discrete location on a solid support. In some embodiments, methods further comprise counting binding events. In some embodiments, methods further comprise counting fluorescent transition events. In some embodiments, methods further comprise measuring the dwell time of binding events. In some embodiments, methods further comprise identifying a candidate signal from a time-dependent fluorescence signal comprising at least 5 transitions during the acquisition time. In some embodiments, methods further comprise calculating a kinetic parameter, calculating a distribution of the number of transitions, and/or calculating a parameter characterizing a distribution. In some embodiments, methods comprise using pattern recognition to process said fluorescence transition data. In some embodiments, methods further comprise statistically analyzing said fluorescence transition data. In some embodiments, methods comprise detecting the analyte using said fluorescence transition data.

Additional embodiments relate to systems for detecting an analyte. In some embodiments, systems comprise a biosensor comprising a capture probe moiety and a query probe moiety, wherein said capture probe moiety comprises a first label and said query probe moiety comprises a second label; and a component configured to record a fluorescence signal indicating the association and dissociation of the query probe with the analyte as a function of time. In some embodiments, systems further comprise a solid support. In some embodiments, systems further comprise a decoy moiety. In some embodiments, systems further comprise a fluorescence microscope. In some embodiments, systems further comprise an excitation source. In some embodiments, systems further comprise an emission detector. In some embodiments, systems further comprise a computer to record and/or analyze fluorescence transition data. In some embodiments, systems further comprise an analyte.

In some embodiments, the capture probe moiety comprises an antibody. In some embodiments, the capture probe moiety comprises a nucleic acid. In some embodiments, the query probe moiety comprises an antibody. In some embodiments, the query probe moiety comprises a nucleic acid. In some embodiments, the decoy moiety comprises an antibody. In some embodiments, the decoy moiety comprises a nucleic acid.

The technology provides uses of biosensors and related methods and systems. In some embodiments, the technology provides use of a biosensor as described herein to detect an analyte. In some embodiments, the technology provides use of a method as described herein to detect an analyte. In some embodiments, the technology provides use of a system as described herein to detect an analyte.

Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings:

FIG. 1A is a schematic drawing showing an embodiment of a kinetic biosensor is described herein. The biosensor comprises several modules: a capture probe (C) for stable binding of the target analyte (T) via a first epitope; a query probe (Q) for reversible kinetic probing (e.g., transient binding) of a second, distinct epitope of T; labels L1 and L2 whose change in relative distance as a function of time report on the presence and identity of the analyte; an optional decoy (D) moiety to compete with T for binding of Q; and an optional anchor moiety (A) for immobilization to a surface or other phase boundary for ease of detection. Upon stable binding of T to C, the complex undergoes a series of transitions between two states: State 1, in which both Q and C are bound to T, and their labels are thus held in close proximity; and State 2, in which only C is bound to T, resulting in a measurably larger distance between labels L1 and L2. In some embodiments, the relative durations of States 1 and 2 are controlled by the connectivity between the modules, the stability of binding of Q to T, and/or the presence and nature of the decoy module D. FIG. 1B is a drawing showing the data traces observed in experiments using the biosensor of FIG. 1a. In absence of analyte, the sensor predominantly occupies state 2. Upon binding of target analyte T, the sensor enters a distinct kinetic pattern of alternation between States 1 and 2. Upon binding of a spurious analyte S (distinct from T but with some affinity for Q and C) a different pattern of alternation between States 1 and 2 is observed, permitting discrimination between T and S.

FIG. 2A to FIG. 2C shows exemplary embodiments and variations on the technology. FIG. 2A shows a schematic drawing of an embodiment of a biosensor constructed using a complex of nucleic acid sequences that recruit a target nucleic acid (e.g., DNA or RNA). In this case, the query and capture probes are low- and high-affinity hybridization probes with partial complementarity to the target T, and the Decoy is a nucleic acid sequence that mimics the portion of T that binds to Q. FIG. 2B shows an embodiment of a biosensor in which the query and capture probes are low- and high-affinity antibodies, respectively. FIG. 2C shows an embodiment of a biosensor in which the decoy D is not covalently linked to the rest of the biosensor components, but is introduced in trans.

FIG. 3A to FIG. 3C shows data collected from an experimental demonstration of an embodiment of the biosensor. FIG. 3A shows an embodiment of a biosensor design comprising three nucleic acid strands in addition to the target nucleic acid (T), the microRNA hsa-miR-141. The capture (C) and query (Q) probes are long and short nucleic acid sequences that bind to adjacent complementary regions on T, resulting in juxtaposition of the labels Alexa Fluor 647 (AF647, L1) and Cy3 (L2) when both C and Q are bound to T. The juxtaposition of Cy3 and AF647 permits Forster resonance energy transfer (FRET) between the two dyes, and as a result nearly 100% of fluorescent signal from the complex is emitted from AF647. When Q is not bound to T, Q instead binds to decoy sequence D, resulting in low FRET efficiency and nearly 100% of fluorescent from the complex is emitted from Cy3. The capture (C) sequence incorporates locked nucleic acid (LNA) modifications for greater stability of target binding by the C probe. FIG. 3B shows FRET measurement of a single biosensor molecule with hsa-miR-141 bound. Data are shown on the plot as a FRET ratio, which is calculated according to A/(A+D), where A is the apparent intensity of fluorescence emission from the acceptor fluorophore and D is the apparent intensity of fluorescence emission from the donor fluorophore. Repeated transitions between a high-FRET state (mostly Cy5 emission) and a low-FRET state (mostly Cy3 emission) are seen, for example, at t=14 seconds and t=16 seconds, permitting kinetic characterization of the interaction with T and thus providing a clear signature of the presence of T. The configurations of the biosensor giving rise to the high-FRET and low-FRET states are shown at the right with dotted lines connecting the signal with the configurations of the biosensor. FIG. 3C shows a FRET measurement in the absence of T, showing only the low-FRET state and thus indicating the absence of T.

FIG. 4A and FIG. 4B shows data collected from an experimental demonstration of an embodiment of the biosensor comprising three nucleic acid strands in addition to the target nucleic acid, miR-141. Schematics on the left indicate the composition of the biosensor (e.g., a biosensor comprising a decoy (bottom schematic) or a decoy-free biosensor (e.g., a biosensor not comprising a decoy) (top schematic)). Plots in the middle show representative single-molecule FRET trajectories from individual biosensors, while plots on the right are histograms of FRET Ratios observed in 36 biosensors (from each condition) exhibiting at least one data point of non-zero FRET efficiency. As used herein, the term “FRET Ratio” is calculated according to A/(A+D), where A is the apparent intensity of fluorescence emission from the acceptor fluorophore and D is the apparent intensity of fluorescence emission from the donor fluorophore. FIG. 4A depicts data resulting from single-molecule FRET measurements of a biosensor in which a decoy (D) is absent, resulting in a stable FRET efficiency close to 1. FIG. 4B depicts data from single-molecule FRET measurements of a biosensor in which a 7-nucleotide decoy sequence is present, resulting in dynamic transitions between FRET efficiencies of approximately 1 and 0.1. Thus, the presence of the decoy results in FRET transitions that are not observed in the biosensor without the decoy.

FIG. 5 is a schematic drawing showing the operating principles of embodiments of the kinetic detection technology provided herein for detecting a cancer-associated miRNA (miR-141) as visualized by TIRF microscopy. As shown by the data presented in the figure, the sensor undergoes rapid transitions between high-FRET and low-FRET states in the presence of the target miRNA. In contrast, the data indicate that only the low-FRET state (e.g., high donor fluorescence and low acceptor fluorescence) is observed in the absence of the target.

FIG. 6 is a bar plot showing the change in median lifetimes of the high-FRET and low-FRET states as a function of the length of the spacer of the decoy (“competitor”) and spacer of the query probe (“loop length”) in nucleotides (nt). The sensor comprising a 6-nt spacer for the decoy (“competitor”) and 18-nt spacer for the query probe (“query arm loop”) was observed to undergo rapid transitions between high-FRET and low-FRET states having similar lifetimes. All kinetics are reported in the presence of the target RNA, miR-141.

FIG. 7A shows single-molecule FRET traces of donor (blue) and acceptor (red) fluorescence of single miR-141-bound sensors (6-nt decoy spacer arm, 18-nt query spacer arm loop) in varying concentrations of formamide.

FIG. 7B shows the lifetimes of the high-FRET and low-FRET states as a function of formamide concentration.

FIG. 7C is a histogram of N_{b+d}, the number of binding and dissociation events per molecule, constructed from only the first 10 seconds of time traces collected in 10% formamide.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology relating to detecting analytes and particularly, but not exclusively, to methods, compositions, and systems for detecting, characterizing, identifying, and/or quantifying analytes.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes only and are not to be construed as limiting the described subject matter in any way.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the terms “about”, “approximately”, “substantially”, and “significantly” are understood by persons of ordinary skill in the art and will vary to some extent on the context in which they are used. If there are uses of these terms that are not clear to persons of ordinary skill in the art given the context in which they are used, “about” and “approximately” mean plus or minus less than or equal to 10% of the particular term and “substantially” and “significantly” mean plus or minus greater than 10% of the particular term.

As used herein, the terms “subject” and “patient” refer to any organisms including plants, microorganisms, and animals (e.g., mammals such as dogs, cats, livestock, and humans).

As used herein, the term “analyte” refers to a substance to be tested, assayed, detected, imaged, characterized, described, and/or quantified. Exemplary analytes include, but are not limited to, molecules, atoms, ions, biomolecules (e.g., nucleic acids (e.g., DNA, RNA) as described and as defined herein), polypeptides (e.g., peptides, proteins, glycoproteins), carbohydrates, lipids, amino acids, small molecules (drugs, bioactive agents, toxins, cofactors, metabolites), etc.

The term “sample” in the present specification and claims is used in its broadest sense. In some embodiments, a sample is or comprises an animal cell or tissue. In some embodiments, a sample includes a specimen or a culture (e.g., a microbiological culture) obtained from any source, as well as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the present technology.

As used herein, a “biological sample” refers to a sample of biological tissue or fluid. For instance, a biological sample may be a sample obtained from an animal (including a human); a fluid, solid, or tissue sample; as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagomorphs, rodents, etc. Examples of biological samples include sections of tissues, blood, blood fractions, plasma, serum, urine, or samples from other peripheral sources or cell cultures, cell colonies, single cells, or a collection of single cells. Furthermore, a biological sample includes pools or mixtures of the above mentioned samples. A biological sample may be provided by removing a sample of cells from a subject, but can also be provided by using a previously isolated sample. For example, a tissue sample can be removed from a subject suspected of having a disease by conventional biopsy techniques. In some embodiments, a blood sample is taken from a subject. A biological sample from a patient means a sample from a subject suspected to be affected by a disease.

Environmental samples include, e.g., environmental material such as surface matter, soil, water, and industrial samples, as well as samples obtained from food and dairy processing instruments, apparatus, equipment, utensils, disposable and non-disposable items. These examples are not to be construed as limiting the sample types applicable to the present invention.

As used herein, the term “biofluid” refers to a biological fluid (e.g., a body fluid, a bodily fluid). For example, in some embodiments, a biofluids is an excretion (e.g., urine, sweat, exudate (e.g., including plant exudate)) and in some embodiments a biofluid is a secretion (e.g., breast milk, bile). In some embodiments, a biofluid is obtained using a needle (e.g., blood, cerebrospinal fluid, lymph). In some embodiments, a biofluid is produced as a result of a pathological process (e.g., a blister, cyst fluid). In some embodiments, a biofluid is derived from another biofluid (e.g., plasma, serum).

Exemplary biofluids include, but are not limited to, amniotic fluid, aqueous humor, vitreous humor, bile, blood, blood plasma, blood serum, breast milk, cerebrospinal fluid, cerumen (earwax), chyle, chime, endolymph, perilymph, exudates, feces, female ejaculate, gastric acid, gastric juice, lymph, mucus (e.g., nasal drainage, phlegm), pericardial fluid, peritoneal fluid, pleural fluid, pus, rheum, saliva, sebum (e.g., skin oil), serous fluid, semen, smegma, sputum, synovial fluid, sweat, tears, urine, vaginal secretion, and vomit.

The term “label” as used herein refers to any atom, molecule, molecular complex (e.g., metal chelate), or colloidal particle (e.g., quantum dot, nanoparticle, microparticle, etc.) that can be used to provide a detectable (e.g., quantifiable) effect, and that can be attached to a nucleic acid or protein. Labels include, but are not limited to, dyes (e.g., optically-detectable labels, fluorescent dyes or moieties, etc.); radiolabels such as ³²P; binding moieties such as biotin; haptens such as digoxigenin; luminogenic, phosphorescent, optically-detectable, or fluorogenic moieties; mass tags; and fluorescent dyes alone or in combination with moieties that can suppress or shift emission spectra by fluorescence resonance energy transfer (FRET). Labels may provide signals detectable by fluorescence, luminescence, radioactivity, colorimetry, gravimetry, X-ray diffraction or absorption, magnetism, enzymatic activity, characteristics of mass or behavior affected by mass (e.g., MALDI time-of-flight mass spectrometry; fluorescence polarization), and the like. A label may be a charged moiety (positive or negative charge) or, alternatively, may be charge neutral. Labels can include or consist of nucleic acid or protein sequence, so long as the sequence comprising the label is detectable.

As used herein the term “fluorophore” will be understood to refer to both fluorophores and luminophores and chemical agents that quench fluorescent or luminescent emissions. Further, as used herein, a “fluorophore” refers to any species possessing a fluorescent property when appropriately stimulated. The stimulation that elicits fluorescence is typically illumination; however, other types of stimulation (e.g., collisional) are also considered herein. The terms “fluorophore”, “fluor, “fluorescent moiety”, “fluorescent dye, and fluorescent group” are used interchangeably.

As used herein a “FRET pair” refers to an acceptor and donor fluorophore wherein the emission wavelength of the donor fluorophore overlaps with the excitation wavelength of the acceptor fluorophore.

“Support” or “solid support”, as used herein, refers to a surface or matrix on or in which an analyte may be concentrated and/or immobilized, e.g., a surface to which an analyte may be covalently or noncovalently attached or in, or on which an analyte may be partially or completely embedded so that the analytes are largely or entirely prevented from diffusing freely or moving with respect to one another. In some embodiments, the support comprises a moiety that interacts with an anchor moiety.

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino, locked nucleic acid (LNA), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand.

The term “nucleotide analog” as used herein refers to modified or non-naturally occurring nucleotides including but not limited to analogs that have altered stacking interactions such as 7-deaza purines (e.g., 7-deaza-dATP and 7-deaza-dGTP); base analogs with alternative hydrogen bonding configurations (e.g., such as Iso-C and Iso-G and other non-standard base pairs described in U.S. Pat. No. 6,001,983 to S. Benner and herein incorporated by reference); non-hydrogen bonding analogs (e.g., non-polar, aromatic nucleoside analogs such as 2,4-difluorotoluene, described by B. A. Schweitzer and E. T. Kool, J. Org. Chem., 1994, 59, 7238-7242, B. A. Schweitzer and E. T. Kool. J. Am. Chem. Soc., 1995, 117, 1863-1872; each of which is herein incorporated by reference); “universal” bases such as 5-nitroindole and 3-nitropyrrole; and universal purines and pyrimidines (such as “K” and “P” nucleotides, respectively; P. Kong, et al., Nucleic Acids Res., 1989, 17, 10373-10383, P. Kong et al., Nucleic Acids Res., 1992, 20, 5149-5152). Nucleotide analogs include nucleotides having modification on the sugar moiety, such as dideoxy nucleotides and 2′-O-methyl nucleotides. Nucleotide analogs include modified forms of deoxyribonucleotides as well as ribonucleotides.

“Peptide nucleic acid” means a DNA mimic that incorporates a peptide-like polyamide backbone.

As used herein, the terms “complementary” or “complementarity” are used in reference to polynucleotides (e.g., a sequence of nucleotides such as an oligonucleotide capture probe, query probe or a target analyte that is a nucleic acid) related by the base-pairing rules. For example, for the sequence “5′-A-G-T-3” is complementary to the sequence “3′-T-C-A-5′.” Complementarity may be “partial.” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

In some contexts, the term “complementarity” and related terms (e.g., “complementary”, “complement”) refers to the nucleotides of a nucleic acid sequence that can bind to another nucleic acid sequence through hydrogen bonds, e.g., nucleotides that are capable of base pairing, e.g., by Watson-Crick base pairing or other base pairing. Nucleotides that can form base pairs, e.g., that are complementary to one another, are the pairs: cytosine and guanine, thymine and adenine, adenine and uracil, and guanine and uracil. The percentage complementarity need not be calculated over the entire length of a nucleic acid sequence. The percentage of complementarity may be limited to a specific region of which the nucleic acid sequences that are base-paired, e.g., starting from a first base-paired nucleotide and ending at a last base-paired nucleotide. The complement of a nucleic acid sequence as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids of the present invention and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

Thus, in some embodiments, “complementary” refers to a first nucleobase sequence that is at least 60%, 65%, 70%, 75%, 80%, 85%, 90%, 95%, 97%, 98%, or 99% identical to the complement of a second nucleobase sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases, or that the two sequences hybridize under stringent hybridization conditions. “Fully complementary” means each nucleobase of a first nucleic acid is capable of pairing with each nucleobase at a corresponding position in a second nucleic acid. For example, in certain embodiments, an oligonucleotide wherein each nucleobase has complementarity to a nucleic acid has a nucleobase sequence that is identical to the complement of the nucleic acid over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases.

“Mismatch” means a nucleobase of a first nucleic acid that is not capable of pairing with a nucleobase at a corresponding position of a second nucleic acid.

The term “domain” when used in reference to a polypeptide refers to a subsection of the polypeptide which possesses a unique structural and/or functional characteristic; typically, this characteristic is similar across diverse polypeptides. The subsection typically comprises contiguous amino acids, although it may also comprise amino acids which act in concert or which are in close proximity due to folding or other configurations. Examples of a protein domain include transmembrane domains, glycosylation sites, etc.

The term “gene” refers to a nucleic acid (e.g., DNA or RNA) sequence that comprises coding sequences necessary for the production of an RNA, or a polypeptide or its precursor (e.g., proinsulin). A functional polypeptide can be encoded by a full length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified,” “mutant,” “variant”, or “polymorphic” refers to a gene or gene product that displays modifications in sequence and or functional properties (e.g., altered characteristics) when compared to the wild-type gene or gene product. It is noted that naturally-occurring mutants can be isolated; these are identified by the fact that they have altered characteristics when compared to the wild-type gene or gene product. Thus, the terms “variant” and “mutant” when used in reference to a nucleotide sequence refer to an nucleic acid sequence that differs by one or more nucleotides from another, usually related nucleotide acid sequence. A “variation” is a difference between two different nucleotide sequences; in some embodiments, one sequence is a reference sequence.

The term “allele” refers to different variations in a gene; the variations include but are not limited to variants and mutants, polymorphic loci and single nucleotide polymorphic loci, frameshift and splice mutations. An allele may occur naturally in a population, or it might arise during the lifetime of any particular individual of the population.

As used herein, the term “hybridization” is used in reference to the pairing of complementary nucleic acids. Hybridization and the strength of hybridization (e.g., the strength of the association between the nucleic acids) is influenced by such factors as the degree of complementary between the nucleic acids, stringency of the conditions involved, and the Tₘ of the formed hybrid. “Hybridization” methods involve the annealing of one nucleic acid to another, complementary nucleic acid, e.g., a nucleic acid having a complementary nucleotide sequence. The ability of two polymers of nucleic acid containing complementary sequences to find each other and anneal through base pairing interaction is a well-recognized phenomenon. The initial observations of the “hybridization” process by Marmur and Lane. Proc. Natl. Acad. Sci. USA 46:453 (1960) and Doty et al., Proc. Natl. Acad. Sci. USA 46:461 (1960) have been followed by the refinement of this process into an essential tool of modern biology.

As used herein, the term “Tm” is used in reference to the “melting temperature.” The melting temperature is the temperature at which a population of double-stranded nucleic acid molecules becomes half dissociated into single strands. Several equations for calculating the Tₘ of nucleic acids are well known in the art. As indicated by standard references, a simple estimate of the Tₘ value may be calculated by the equation: Tₘ=81.5+0.41*(% G+C), when a nucleic acid is in aqueous solution at 1 M NaCl (see, e.g., Anderson and Young, Quantitative Filter Hybridization, in Nucleic Acid Hybridization (1985). Other references (e.g., Allawi and SantaLucia, Biochemistry 36: 10581-94 (1997) include more sophisticated computations which account for structural, environmental, and sequence characteristics to calculate Tₘ. For example, in some embodiments these computations provide an improved estimate of Tₘ for short nucleic acid probes and targets (e.g., as used in the examples).

As used herein, the term “acquisition time”, “observation period”, “observation time”, and similar phrases refer to the amount of time during which an embodiment of the technology is used to observe a sample and/or record data to detect an analyte, if present, in a sample. In particular, in some embodiments, the “acquisition time”, “observation period”, “observation time”, and similar phrases refer to the amount of time during which query probe binding events are detected, recorded, and/or counted. In some embodiments, the “acquisition time”, “observation period”, “observation time”, and similar phrases refer to an amount of time that is tens to hundreds to thousands of seconds, e.g., 5, 10, 15, 20, 25, 30, 35, 40, 45, 50, 55, or 60 seconds; e.g., 5, 10, 15, 20, 25, 30, 35, 40, 45, 50, 55, or 60 minutes; e.g., 1, 1.5, 2, 2.5, or 3 hours. In some embodiments, the “acquisition time”, “observation period”, “observation time”, and similar phrases refer to an amount of time that is approximately 1 to 10 seconds to 1 to 10 minutes (e.g., approximately 1 to 100 seconds, e.g., 1, 2, 3, 4, 5, 6, 7, 8, 9, 10, 11, 12, 13, 14, 15, 16, 17, 18, 19, 20, 30, 40, 50, 60, 70, 80, 90, or 100 seconds, e.g., 1 to 100 minutes, e.g., 1, 2, 3, 4, 5, 6, 7, 8, 9, 10, 11, 12, 13, 14, 15, 16, 17, 18, 19, 20, 30, 40, 50, 60, 70, 80, 90, or 100 minutes. In some embodiments, the acquisition time is 0.1-20.0 seconds (e.g., 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, or 20.0 seconds).

As used herein, the term “measurement temperature” refers to the temperature at which an embodiment of the technology is used to observe a sample and/or record data to detect an analyte, if present, in a sample. In some embodiments, the “measurement temperature” is controlled using active heating and cooling (e.g., a heat pump, Peltier device, water bath, or other technology known in the art to maintain the temperature of a sample, e.g., within 0.1 to 0.5 to 1.0° C. of a set temperature). In some embodiments, the measurement temperature is 10 to 110° C. (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, or 110° C.).

The terms “protein” and “polypeptide” refer to compounds comprising amino acids joined via peptide bonds and are used interchangeably. A “protein” or “polypeptide” encoded by a gene is not limited to the amino acid sequence encoded by the gene, but includes post-translational modifications of the protein. Where the term “amino acid sequence” is recited herein to refer to an amino acid sequence of a protein molecule, “amino acid sequence” and like terms such as “polypeptide” or “protein” are not meant to limit the amino acid sequence to the complete, native amino acid sequence associated with the recited protein molecule. Furthermore, an “amino acid sequence” can be deduced from the nucleic acid sequence encoding the protein. Conventional one and three-letter amino acid codes are used herein as follows—Alanine: Ala, A; Arginine: Arg, R; Asparagine: Asn, N; Aspartate: Asp, D; Cysteine: Cys, C; Glutamate: Glu, E: Glutamine: Gln, Q; Glycine: Gly, G; Histidine: His, H; Isoleucine: Ile, I; Leucine: Leu, L; Lysine: Lys, K; Methionine: Met, M; Phenylalanine: Phe, F; Proline: Pro, P; Serine: Ser, S; Threonine: Thr, T; Tryptophan: Trp, W; Tyrosine: Tyr, Y; Valine: Val, V. As used herein, the codes Xaa and X refer to any amino acid.

The terms “variant” and “mutant” when used in reference to a polypeptide refer to an amino acid sequence that differs by one or more amino acids from another, usually related polypeptide.

As used herein, the term “melting” when used in reference to a nucleic acid refers to the dissociation of a double-stranded nucleic acid or region of a nucleic acid into a single-stranded nucleic acid or region of a nucleic acid.

As used herein, a “query probe” or “reader probe” is any entity (e.g., molecule, biomolecule, etc.) that recognizes an analyte (e.g., binds to an analyte, (e.g., binds specifically to an analyte)). In exemplary embodiments, the query probe is a protein that recognizes an analyte. In some other exemplary embodiments, the query probe is a nucleic acid that recognizes an analyte. For example, in some embodiments the query probe comprises, e.g., DNA, RNA, a nucleic acid comprising DNA and RNA, a nucleic acid comprising modified bases and/or modified linkages between bases such as, e.g., a nucleic acid as described hereinabove. In some embodiments, the nucleic acid query probe comprises a nucleic acid aptamer. In some embodiments, the query probe is labeled, e.g., with a detectable label such as, e.g., a fluorescent moiety as described herein. In some embodiments, the query probe comprises more than one type of molecule (e.g., more than one of a protein, a nucleic acid, a chemical linker or a chemical moiety). In some embodiments, the query probe is labeled with a member of a FRET pair. In some embodiments, the query probe is labeled with a quencher. In some embodiments, a query probe binds to an analyte with moderate to low thermodynamic stability (e.g., with a melting temperature within 10 degrees C. of the measurement temperature, or an average lifetime no more than ⅕th as long as the observation period). In some embodiments, the query probe comprises a spacer arm comprising 10-30 nt (e.g., 10, 11, 12, 13, 14, 15, 16, 17, 18, 19, 20, 21, 22, 23, 24, 25, 26, 27, 28, 29, or 30 nt.). In some embodiments, the query probe comprises 5-50 nt (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nt).

Embodiments of the technology comprise a query probe (e.g., a detectably labeled query probe) that binds transiently and repeatedly to the analyte, e.g., a query probe that binds to and dissociates from the target analyte several (e.g., greater than 1, 2, 3, 4, 5, 6, 7, 8, 9, 10, or more) times per observation window. In some embodiments, the query probe has a dissociation constant (K_{D}) for the analyte of larger than approximately 1 nanomolar (e.g., greater than 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 or more nanomolar) under the assay conditions. In some embodiments, the query probe has a binding and/or a dissociation constant for the analyte that is larger than approximately 1 min⁻¹ (e.g., greater than 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 or more min).

The technology is not limited in the query probe. In some embodiments, the query probe is an antibody or antibody fragment. In some embodiments, the query probe is a low-affinity antibody or antibody fragment. In some embodiments, the query probe is a nanobody, a DNA-binding protein or protein domain, a methylation binding domain (MBD), a kinase, a phosphatase, an acetylase, a deacetylase, an enzyme, or a polypeptide. In some embodiments, the query probe is an oligonucleotide that interacts with the target analyte. For example, in some embodiments the query probe is an oligonucleotide that hybridizes to the target analyte to form a duplex that has a melting temperature that is within approximately 10 degrees Celsius of the temperature at which the observations are made (e.g., approximately 7-12 nucleotides for observation that is performed at room temperature). In some embodiments, the query probe is a mononucleotide. In some embodiments, the query probe is a small organic molecule (e.g., a molecule having a molecular weight that is less than approximately 2000 daltons, e.g., less than 2100, 2050, 2000, 1950, 1900, 1850, 1800, 1750, 1700, 1650, 1600, 1550, 1500 daltons, or less). In some embodiments, the query probe is a pharmaceutical agent, e.g., a drug or other bioactive molecule. In some embodiments, the query probe is a metal ion complex. In some embodiments, the query probe is a methyl-binding domain (e.g., MBD1). In some embodiments, the query probe is labeled with a detectable label as described herein. In some embodiments, the query probe is covalently linked to the detectable label. In some embodiments, the query probe is indirectly and/or non-covalently linked and/or associated with the detectable label. In some embodiments, the detectable label is fluorescent.

In some embodiments, the query probe is a mouse monoclonal antibody.

In some embodiments in which the analyte comprises a carbohydrate or polysaccharide, the query probe comprises a carbohydrate-binding protein such as a lectin or a carbohydrate-binding antibody.

As used herein, an “event” refers to an instance of a query probe binding to an analyte or an instance of query probe dissociation from an analyte, e.g., as measured by monitoring a detectable property indicating the binding of a query probe to an analyte and/or the dissociation of a query probe from an analyte.

As used herein, the term “decoy moiety” is any entity (e.g., molecule, biomolecule, etc.) that binds to the query probe and that is not the analyte. In some embodiments, the decoy moiety competes with the query probe for binding to the analyte and prolongs the time the query probe is in a state not bound to the analyte. Accordingly, in some embodiments the decoy moiety allows for control of the interaction (e.g., association and dissociation) of the query probe with the analyte, e.g., provides control over the kinetics of the query probe switching between “State 1”, in which the query probe is associated with the analyte and “State 2”, in which the query probe is not associated with the analyte.

In some embodiments, the decoy moiety is an analyte mimic. In some embodiments, the decoy moiety and the query probe are directly connected (e.g., covalently (e.g., by a bond, linker, etc.)). In some embodiments, the decoy moiety and the query probe are indirectly connected (e.g., by non-covalent interaction). In embodiments in which the query probe and decoy moiety are directly or indirectly connected, the decoy moiety is provided “in cis” with respect to the query probe. In some embodiments, the decoy moiety and the query probe are not connected to one another (e.g., the decoy moiety is present “in trans” with respect to the query probe). In various embodiments, the decoy moiety binds less strongly to the query probe or binds more strongly to the query probe than the analyte binds to the query probe. In some embodiments, the decoy moiety binds to the query probe with approximately the same affinity as the query probe binds to the analyte. In exemplary embodiments, the decoy moiety is a protein that binds to the query probe. In some other exemplary embodiments, the decoy moiety is a nucleic acid that binds to the query probe. For example, in some embodiments the decoy moiety comprises, e.g., DNA, RNA, a nucleic acid comprising DNA and RNA, a nucleic acid comprising modified bases and/or modified linkages between bases such as, e.g., a nucleic acid as described hereinabove. In some embodiments, the decoy moiety comprises a nucleic acid aptamer. In some embodiments, the decoy moiety is labeled, e.g., with a detectable label such as, e.g., a fluorescent moiety as described herein. In some embodiments, the decoy moiety comprises more than one type of molecule (e.g., more than one of a protein, a nucleic acid, a chemical linker or a chemical moiety). In some embodiments, the decoy moiety is labeled with a member of a FRET pair. In some embodiments, the decoy moiety is labeled with a quencher. In some embodiments, a decoy moiety binds to the query probe with moderate to low thermodynamic stability (e.g., with a melting temperature within 10 degrees C. of the measurement temperature, or an average lifetime no more than ⅕th as long as the observation period). In some embodiments, the decoy moiety is a nucleic acid comprising 4-20 nt (e.g., 4, 5, 6, 7, 8, 9, 10, 11, 12, 13, 14, 15, 16, 17, 18, 19, or 20 nt).

As used herein, a “capture probe” is any entity (e.g., molecule, biomolecule, etc.) that recognizes an analyte (e.g., binds to an analyte, e.g., binds specifically to an analyte). In some embodiments, a capture probe links the analyte to a solid support. In exemplary embodiments, the capture probe is a protein that recognizes an analyte. In some other exemplary embodiments, a capture probe is a nucleic acid that recognizes an analyte. For example, in some embodiments the capture probe comprises, e.g., DNA, RNA, a nucleic acid comprising DNA and RNA, a nucleic acid comprising modified bases and/or modified linkages between bases such as, e.g., a nucleic acid as described hereinabove. In some embodiments, the nucleic acid capture probe comprises a nucleic acid aptamer. In some embodiments, the capture probe is labeled, e.g., with a detectable label such as, e.g., a fluorescent moiety as described herein. In some embodiments, the capture probe comprises more than one type of molecule (e.g., more than one of a protein, a nucleic acid, a chemical linker or a chemical moiety). In some embodiments, the capture probe is labeled with a member of a FRET pair. In some embodiments, the capture probe is labeled with a quencher. In some embodiments, the capture probe binds an analyte with high thermodynamic stability (e.g., with a melting temperature more than 10 degrees C. above the measurement temperature or an average lifetime more than 10 times longer than the observation period). In some embodiments, the capture probe comprises an anchor moiety. In some embodiments, the capture probe is directly (e.g., covalently) connected to an anchor moiety. In some embodiments, the capture probe is indirectly (e.g., non-covalently) connected to an anchor moiety.

Embodiments of the technology comprise capture of an analyte. In some embodiments, the analyte is captured and immobilized. In some embodiments, the analyte is stably attached to a solid support. In some embodiments, the solid support is immobile relative to a bulk liquid phase contacting the solid support. In some embodiments, the solid support is diffusible within a bulk liquid phase contacting the solid support.

In some embodiments, stable attachment of the target analyte to a surface or other solid substrate is provided by a high-affinity or irreversible interaction (e.g., as used herein, an “irreversible interaction” refers to an interaction having a dissociation half-life longer than the observation time, e.g., in some embodiments, a time that is 1 to 10 minutes (e.g., 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or 600 seconds, or longer). The technology is not limited in the components and/or methods used for capture of the analyte. For example, the stable attachment is provided by a variety of methods, including but not limited to one or more of the following. In some embodiments, an irreversible interaction in an interaction having a high thermodynamic stability (e.g., with a melting temperature more than 10 degrees C. above the measurement temperature or an average lifetime more than 10 times longer than the observation period).

In some embodiments, an analyte is immobilized by a surface-bound capture probe with a dissociation constant (K_{D}) for the analyte smaller than approximately 1 nanomolar (nM) (e.g., less than 1.5, 1.4, 1.3, 1.2, 1.1, 1.0, 0.9, 0.8, 0.7, 0.6, 0.5 nanomolar) and a dissociation rate constant for the analyte that is smaller than approximately 1 min⁻¹ (e.g., less than approximately 1.5, 1.4, 1.3, 1.2, 1.1, 1.0, 0.9, 0.8, 0.7, 0.6, 0.5 min⁻¹). Exemplary surface-bound capture probes include, e.g., an antibody, antibody fragment, nanobody, or other protein; a high-affinity DNA-binding protein or ribonucleoprotein complex such as Cas9, dCas9, Cpf1, transcription factors, or transcription activator-like effector nucleases (TALENs); an oligonucleotide; a small organic molecule; or a metal ion complex.

In some embodiments, an analyte is immobilized by direct noncovalent attachment to a surface (e.g., by interactions between the analyte and the surface, e.g., a glass surface or a nylon, nitrocellulose, or polyvinylidene difluoride membrane).

In some embodiments, an analyte is immobilized by chemical linking (e.g., by a covalent bond) of the analyte to the solid support. In some embodiments, the analyte is chemically linked to the solid support by, e.g., a carbodiimide, a N-hydroxysuccinimide esters (NHS) ester, a maleimide, a haloacetyl group, a hydrazide, or an alkoxyamine. In some embodiments, an analyte is immobilized by radiation (e.g., ultraviolet light)-induced cross-linking of the target analyte to the surface and/or to a capture probe attached to the surface. In some embodiments, the capture probe is a rabbit monoclonal antibody. In some embodiments in which the analyte comprises a carbohydrate or polysaccharide, the capture probe comprises a carbohydrate-binding protein such as a lectin or a carbohydrate-binding antibody.

Alternatively, instead of immobilizing the target analyte to a solid support that is relatively stationary with respect to a bulk phase that contacts the solid support as described above, some embodiments provide that the target analyte is associated with a freely diffusing particle that diffuses within the bulk fluid phase contacting the freely diffusing particle. Accordingly, in some embodiments, the target analyte is covalently or noncovalently bound to a freely diffusing substrate. In some embodiments, the freely diffusing substrate is, e.g., a colloidal particle (e.g., a particle having a diameter of approximately 10-1000 nm (e.g., 10, 20, 30, 40, 50, 60, 70, 80, 90, 100, 110, 120, 130, 140, 150, 200, 250, 300, 350, 400, 450, 500, 550, 600, 650, 700, 750, 800, 850, 900, 950, or 1000 nm)). In some embodiments, the freely diffusing substrate comprises and/or is made of, e.g., polystyrene, silica, dextran, gold, or DNA origami. In some embodiments, the target analyte is associated with a freely diffusing particle that diffuses slowly relative to the diffusion of the query probe, e.g., the target analyte has a diffusion coefficient that is less than approximately 10% (e.g., less than 15, 14, 13, 12, 11, 10.5, 10.4, 10.3, 10.2, 10.1, 10.0, 9.9, 9.8, 9.7, 9.6, 9.5, or 9.0% or less) of the diffusion coefficient of the query probe.

Furthermore, in some embodiments the target analyte is associated with a freely diffusing particle and the location of the target analyte is observable and/or recordable independently of observing and/or recording query probe binding. For example, in some embodiments a detectable label (e.g., a fluorophore, fluorescent protein, quantum dot) is covalently or noncovalently attached to the target analyte, e.g., for detection and localization of the target analyte. Accordingly, in some embodiments the position of the target analyte and the position of query probe binding events are simultaneously and independently measured.

As used herein, the term “sensitivity” refers to the probability that an assay gives a positive result for the analyte when the sample comprises the analyte. Sensitivity is calculated as the number of true positive results divided by the sum of the true positives and false negatives. Sensitivity is a measure of how well an assay detects an analyte.

As used herein, the term “specificity” refers to the probability that an assay gives a negative result when the sample does not comprise the analyte. Specificity is calculated as the number of true negative results divided by the sum of the true negatives and false positives. Specificity is a measure of how well a method of the present invention excludes samples that do not comprise an analyte from those that do comprise the analyte.

As used herein, the “equilibrium constant” (K_{eq}), the “equilibrium association constant” (Kₐ), and “association binding constant” (or “binding constant” (K_{B}) are used interchangeably for the following binding reaction of A and B at equilibrium:

A+B [symbol] =AB

where A and B are two entities that associate with each other (e.g., capture probe and analyte, query probe and analyte, decoy moiety and query probe) and K_{eq}=[AB]/([A]×[B]). The dissociation constant K_{D}=1/K_{B}. The K_{D} is a useful way to describe the affinity of a one binding partner A for a partner B with which it associates, e.g., the number Kr represents the concentration of A or B that is required to yield a significant amount of AB. K_{eq}=k_{off}/kₒₙ; K_{D}=k_{off}/kₒₙ.

As used herein, a “significant amount” of the product of two entities that associate with each other, e.g., formation of AB from A and B according to the equation above, refers to a concentration of AB that is equal to or greater than the free concentration of A or B, whichever is smaller.

As used herein, “nanomolar affinity range” refers to the association of two components that has an equilibrium dissociation constant K_{D} (e.g., ratio of k_{off}/kₒₙ) in the nanomolar range, e.g., a dissociation constant (K_{D}) of 1×10⁻¹⁰ to 1×10⁻⁵ M (e.g., in some embodiments 1×10⁻⁹ to 1×10⁻⁶ M). The dissociation constant has molar units (M). The smaller the dissociation constant, the higher the affinity between two components (e.g., capture probe and analyte; query probe and analyte; query probe and decoy moiety).

As used herein, a “weak affinity” or “weak binding” or “weak association” refers to an association having a K_{D} of approximately 100 nanomolar (e.g., approximately 10, 20, 30, 40, 50, 60, 70, 80, 90, 100, 110, 120, 130, 140, 150, 160, 170, 180, 190, 200, 300, 400, or 500 nanomolar) and/or, in some embodiments, in the range of 1 nanomolar to 10 micromolar.

The terms “specific binding” or “specifically binding” when used in reference to the interaction of two components A and B that associate with one another refers to an association of A and B having a K_{D} that is smaller than the K_{D} for the interaction of A or B with other similar components in the solution, e.g., at least one other molecular species in the solution that is not A or B.

As used herein, the word “presence” or “absence” (or, alternatively, “present” or “absent”) is used in a relative sense to describe the amount or level of a particular entity (e.g., an analyte). For example, when an analyte is said to be “present” in a sample, it means the level or amount of this analyte is above a pre-determined threshold; conversely, when an analyte is said to be “absent” in a sample, it means the level or amount of this analyte is below a pre-determined threshold. The pre-determined threshold may be the threshold for detectability associated with the particular test used to detect the analyte or any other threshold. When an analyte is “detected” in a sample it is “present” in the sample; when an analyte is “not detected” it is “absent” from the sample. Further, a sample in which an analyte is “detected” or in which the analyte is “present” is a sample that is “positive” for the analyte. A sample in which an analyte is “not detected” or in which the analyte is “absent” is a sample that is “negative” for the analyte.

As used herein, an “increase” or a “decrease” refers to a detectable (e.g., measured) positive or negative change in the value of a variable relative to a previously measured value of the variable, relative to a pre-established value, and/or relative to a value of a standard control. An increase is a positive change preferably at least 10%, more preferably 50%, still more preferably 2-fold, even more preferably at least 5-fold, and most preferably at least 10-fold relative to the previously measured value of the variable, the pre-established value, and/or the value of a standard control. Similarly, a decrease is a negative change preferably at least 10%, more preferably 50%, still more preferably at least 80%, and most preferably at least, 90% of the previously measured value of the variable, the pre-established value, and/or the value of a standard control, Other terms indicating quantitative changes or differences, such as “more” or “less,” are used herein in the same fashion as described above.

The term “detection assay” refers to an assay for detecting the presence or absence of an analyte or the activity or effect of an analyte or for detecting the presence or absence of a variant of an analyte.

A “system” denotes a set of components, real or abstract, comprising a whole where each component interacts with or is related to at least one other component within the whole.

In some embodiments the technology comprises an antibody component or moiety, e.g., an antibody or fragments or derivatives thereof. As used herein, an “antibody”, also known as an “immunoglobulin” (e.g., IgG, IgM, IgA, IgD, IgE), comprises two heavy chains linked to each other by disulfide bonds and two light chains, each of which is linked to a heavy chain by a disulfide bond. The specificity of an antibody resides in the structural complementarity between the antigen combining site of the antibody (or paratope) and the antigen determinant (or epitope). Antigen combining sites are made up of residues that are primarily from the hypervariable or complementarity determining regions (CDRs). Occasionally, residues from nonhypervariable or framework regions influence the overall domain structure and hence the combining site. Some embodiments comprise a fragment of an antibody, e.g., any protein or polypeptide-containing molecule that comprises at least a portion of an immunoglobulin molecule such as to permit specific interaction between said molecule and an antigen. The portion of an immunoglobulin molecule may include, but is not limited to, at least one complementarity determining region (CDR) of a heavy or light chain or a ligand binding portion thereof, a heavy chain or light chain variable region, a heavy chain or light chain constant region, a framework region, or any portion thereof. Such fragments may be produced by enzymatic cleavage, synthetic or recombinant techniques, as known in the art and/or as described herein, Antibodies can also be produced in a variety of truncated forms using antibody genes in which one or more stop codons have been introduced upstream of the natural stop site. The various portions of antibodies can be joined together chemically by conventional techniques, or can be prepared as a contiguous protein using genetic engineering techniques.

Fragments of antibodies include, but are not limited to, Fab (e.g., by papain digestion), F(ab′)₂ (e.g., by pepsin digestion), Fab′ (e.g., by pepsin digestion and partial reduction) and Fv or scFv (e.g., by molecular biology techniques) fragments.

A Fab fragment can be obtained by treating an antibody with the protease papaine. Also, the Fab may be produced by inserting DNA encoding a Fab of the antibody into a vector for prokaryotic expression system or for eukaryotic expression system, and introducing the vector into a prokaryote or eukaryote to express the Fab. A F(ab′)₂ may be obtained by treating an antibody with the protease pepsin. Also, the F(ab′)₂ can be produced by binding a Fab′ via a thioether bond or a disulfide bond. A Fab may be obtained by treating F(ab′)₂ with a reducing agent, e.g., dithiothreitol. Also, a Fab′ can be produced by inserting DNA encoding a Fab′ fragment of the antibody into an expression vector for a prokaryote or an expression vector for a eukaryote, and introducing the vector into a prokaryote or eukaryote for its expression. A Fv fragment may be produced by restricted cleavage by pepsin, e.g., at 4° C., and pH 4.0, (a method called “cold pepsin digestion”). The Fv fragment consists of the heavy chain variable domain (V_{H}) and the light chain variable domain (V_{L}) held together by strong noncovalent interaction. A scFv fragment may be produced by obtaining cDNA encoding the V_{H} and V_{L} domains as previously described, constructing DNA encoding scFv, inserting the DNA into an expression vector for prokaryote or an expression vector for eukaryote, and then introducing the expression vector into a prokaryote or eukaryote to express the scFv.

In general, antibodies can usually be raised to any antigen, using the many conventional techniques now well known in the art.

As used herein, the term “conjugated” refers to when one molecule or agent is physically or chemically coupled or adhered to another molecule or agent. Examples of conjugation include covalent linkage and electrostatic complexation. The terms “complexed,” “complexed with,” and “conjugated” are used interchangeably herein.

As used herein, a “stable interaction” or referring to a “stably bound” interaction refers to an association that is relatively persistent under the thermodynamic equilibrium conditions of the interaction. In some embodiments, a “stable interaction” is an interaction between two components having a K_{D} that is smaller than approximately 10⁻⁹ M or, in some embodiments, a K_{D} that is smaller than 10⁻⁸ M. In some embodiments, a “stable interaction” has a dissociation rate constant k_{off} that is smaller than 1 per hour or, in some embodiments, a dissociation rate constant k_{off} that is smaller than 1 per minute. In some embodiments, a “stable interaction” is defined as not being a “transient interaction”. In some embodiments, a “stable interaction” includes interactions mediated by covalent bonds and other interactions that are not typically described by a Kc, value but that involve an average association lifetime between two entities that is longer than approximately 1 minute (e.g., 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 seconds) per each interaction.

In some embodiments, the distinction between a “stable interaction” and a “transient interaction” is determined by a cutoff value of K_{D} and/or k_{off} and/or another kinetic or thermodynamic value describing the associations, wherein the cutoff is used to discriminate between stable and transient interactions that might otherwise be characterized differently if described in absolute terms of a K_{D} and/or k_{off} or another kinetic or thermodynamic value describing the associations. For example, a “stable interaction” characterized by a K_{D} value might also be characterized as a “transient interaction” in the context of another interaction that is even more stable. One of skill in the art would understand other relative comparisons of stable and transient interactions, e.g., that a “transient interaction” characterized by a K_{D} value might also be characterized as a “stable interaction” in the context of another interaction that is even more transient (less stable).

As used herein, the terms “stable interaction”, “stable binding”, and “stable association” are used interchangeably. As used herein, the terms “transient interaction”, “transient binding”, and “transient association” are used interchangeably.

As used herein, “moiety” refers to one of two or more parts into which something may be divided, such as, for example, the various parts of an oligonucleotide, a molecule, a chemical group, a domain, a probe, an “R” group, a polypeptide, etc.

As used herein, in some embodiments a “signal” is a time-varying quantity (e.g., an observable, detectable, and/or measurable quantity) associated with one or more properties of a sample that is assayed, e.g., the binding of a query probe to an analyte and/or dissociation of a query probe from an analyte. A signal can be continuous in the time domain or discrete in the time domain. As a mathematical abstraction, the domain of a continuous-time signal is the set of real numbers (or an interval thereof) and the domain of a discrete-time signal is the set of integers (or an interval thereof). Discrete signals often arise via “digital sampling” of continuous signals. For example, an audio signal consists of a continually fluctuating voltage on a line that can be digitized by reading the voltage level on the line at a regular interval, e.g., every 50 microseconds. The resulting stream of numbers is stored as a discrete-time digital signal. In some embodiments, the signal is recorded as a function of location is space (e.g., x, y coordinates; e.g., x, y, z coordinates). In some embodiments, the signal is recorded as a function of time. In some embodiments, the signal is recorded as a function of time and location.

The term “substantially” as used herein, is a broad term and is used in its ordinary sense, including, but not limited to, being largely but not necessarily wholly that which is specified.

The term “algorithm,” as used herein, is a broad term and is used in its ordinary sense, including, but not limited to, the computational processes (for example, programs) involved in transforming information from one state to another, for example using computer processing.

### DESCRIPTION

Although the disclosure herein refers to certain illustrated embodiments, it is to be understood that these embodiments are presented by way of example and not by way of limitation.

### SiMREPS

As used herein, the term “single-molecule recognition through equilibrium Poisson sampling” and its abbreviation “SiMREPS” refers to an amplification-free, single-molecule detection approach for identifying and counting individual analytes in a sample by kinetic fingerprinting. The technology is described in, e.g., U.S. patent application Ser. No. 14/589,467; Int'l Pat. App. No. PCT/US2015/044650; Int'l Pat. App. No. PCT/US2017/016977; Int'l Pat. App. No. PCT/US2018/021356; U.S. Provisional App. Ser. No. 62/468,578, U.S. Provisional App. Ser. No. 62/692,001, and U.S. Provisional App. Ser. No. 62/598,802, each of which is incorporated herein by reference in its entirety. See also Johnson-Buck et al. (2015) “Kinetic fingerprinting to identify and count single nucleic acids” Nature Biotechnology 33: 730-32, incorporated herein by reference in its entirety).

In brief, the SiMREPS technology comprises directly observing the repeated binding of fluorescent probes to surface-captured analytes (e.g., nucleic acid, protein, etc.), which produces a specific (e.g., for nucleic acid, a sequence-specific) kinetic fingerprint. The kinetic fingerprint identifies the analyte with high-confidence at single-molecule resolution. The kinetic fingerprint overcomes previous technologies limited by thermodynamic specificity barriers and thereby minimizes and/or eliminates false positives. Thus, the SiMREPS technology provides an ultra-high specificity that finds use in detecting, e.g., rare analytes such as rare or low-abundance mutant DNA alleles. Prior work has shown that SiMREPS is capable of single-nucleotide discrimination (see, e.g., Johnson-Buck et al. (2015) “Kinetic fingerprinting to identify and count single nucleic acids” Nat. Biotechnol 33: 730-32; Su et al. (2017) “Single-Molecule Counting of Point Mutations by Transient DNA Binding” SciRep 7: 43824, each of which is incorporated herein by reference).

The technology provides for the detection of target analytes, e.g., in the presence of similar analytes and, in some embodiments, background noise. In some embodiments, signal originating from the transient binding of the query probe to the target analyte is distinguishable from the signal produced by unbound query probe (e.g., by observing, monitoring, and/or recording a localized change in signal intensity during the binding event). In some embodiments, observing the transient binding of the query probe (e.g., a fluorescently labeled query probe) to the target analyte is provided by a technology such as, e.g., total internal reflection fluorescence (TIRF) or near-TIRF microscopy, zero-mode waveguides (ZMWs), light sheet microscopy, stimulated emission depletion (STED) microscopy, or confocal microscopy. In some embodiments, the technology provided herein uses query probes having a fluorescence emission that is quenched when not bound to the target analyte and/or a fluorescence emission that is dequenched when bound to the target analyte.

The technology comprises locating and/or observing the transient binding of a query probe to an analyte within a discrete region of an area and/or a discreet region of a volume that is observed, e.g., at particular spatial coordinates in a plane or a volume. In some embodiments, the error in determining the spatial coordinates of a binding or dissociation event (e.g., due to limited signal, detector noise, or spatial binning in the detector) is small (e.g., minimized, eliminated) relative to the average spacing between immobilized (e.g., surface-bound) target analytes. In some embodiments comprising use of wide-field fluorescence microscopy, measurement errors are minimized and/or eliminated by use of effective detector pixel dimensions in the specimen plane that are not larger than the average distance between immobilized (e.g., surface-bound) target analytes and that many fluorescent photons (in some embodiments, more than 100, e.g., more than 80, 85, 90, 95, 100, 105, 110, 115, 120, 125, or 130 or more) are collected per time point of detection.

In some embodiments, the detectable (e.g., fluorescent) query probe produces a fluorescence emission signal when it is close to the surface of the solid support (e.g., within about 100 nm of the surface of the solid support). When unbound, query probes quickly diffuse and thus are not individually detected; accordingly, when in the unbound state, the query probes produce a low level of diffuse background fluorescence.

Consequently, in some embodiments detection of bound query probes comprises use of total internal reflection fluorescence microscopy (TIRF), HiLo microscopy (see, e.g., US20090084980, EP2300983 B1, WO2014018584 A1, WO2014018584 A1, incorporated herein by reference), confocal scanning microscopy, or other technologies comprising illumination schemes that illuminate (e.g., excite) only those query probe molecules near or on the surface of the solid support. Thus, in some embodiments, only query probes that are bound to an immobilized target near or on the surface produce a point-like emission signal (e.g., a “spot”) that can be confirmed as originating from a single molecule.

In some embodiments, the query probe comprises a fluorescent label having an emission wavelength. Detection of fluorescence emission at the emission wavelength of the fluorescent label indicates that the query probe is bound to an immobilized target analyte. Binding of the query probe to the target analyte is a “binding event”. In some embodiments of the technology, a binding event has a fluorescence emission having a measured intensity greater than a defined threshold. For example, in some embodiments a binding event has a fluorescence intensity that is above the background fluorescence intensity (e.g., the fluorescence intensity observed in the absence of a target analyte). In some embodiments, a binding event has a fluorescence intensity that is at least 1, 2, 3, 4 or more standard deviations above the background fluorescence intensity (e.g., the fluorescence intensity observed in the absence of a target analyte). In some embodiments, a binding event has a fluorescence intensity that is at least 2 standard deviations above the background fluorescence intensity (e.g., the fluorescence intensity observed in the absence of a target analyte). In some embodiments, a binding event has a fluorescence intensity that is at least 1.5, 2, 3, 4, or 5 times the background fluorescence intensity (e.g., the mean fluorescence intensity observed in the absence of a target analyte).

Accordingly, in some embodiments detecting fluorescence at the emission wavelength of the query probe that has an intensity above the defined threshold (e.g., at least 2 standard deviations greater than background intensity) indicates that a binding event has occurred (e.g., at a discrete location on the solid support where a target analyte is immobilized). Also, in some embodiments detecting fluorescence at the emission wavelength of the query probe that has an intensity above the defined threshold (e.g., at least 2 standard deviations greater than background intensity) indicates that a binding event has started. Accordingly, in some embodiments detecting an absence of fluorescence at the emission wavelength of the query probe that has an intensity above the defined threshold (e.g., at least 2 standard deviations greater than background intensity) indicates that a binding event has ended (e.g., the query probe has dissociated from the target analyte). The length of time between when the binding event started and when the binding event ended (e.g., the length of time that fluorescence at the emission wavelength of the fluorescent probe having an intensity above the defined threshold (e.g., at least 2 standard deviations greater than background intensity) is detected) is the dwell time of the binding event. A “transition” refers to the binding and dissociation of a query probe to the target analyte (e.g., an on/off event), e.g., a query probe dissociating from a bound state or a query probe associating with a target analyte from the unbound state.

Methods according to the technology comprise counting the number of query probe binding events that occur at each discrete location (e.g., at a position identified by x, y coordinates) on the solid support during a defined time interval that is the “acquisition time” or “observation period” (e.g., a time interval that is tens to hundreds to thousands of seconds, e.g., 5, 10, 15, 20, 25, 30, 35, 40, 45, 50, 55, or 60 seconds; e.g., 5, 10, 15, 20, 25, 30, 35, 40, 45, 50, 55, or 60 minutes; e.g., 1, 1.5, 2, 2.5, or 3 hours). In some embodiments, the acquisition time is approximately 1 to 10 seconds to 1 to 10 minutes (e.g., approximately 1 to 100 seconds, e.g., 1, 2, 3, 4, 5, 6, 7, 8, 9, 10, 11, 12, 13, 14, 15, 16, 17, 18, 19, 20, 30, 40, 50, 60, 70, 80, 90, or 100 seconds, e.g., 1 to 100 minutes, e.g., 1, 2, 3, 4, 5, 6, 7, 8, 9, 10, 11, 12, 13, 14, 15, 16, 17, 18, 19, 20, 30, 40, 50, 60, 70, 80, 90, or 100 minutes). In some embodiments, the acquisition time is 0.1-20.0 seconds (e.g., 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, or 20.0 seconds).

Further, the length of time the query probe remains bound to the target analyte during a binding event is the “dwell time” of the binding event. The number of binding events detected during the acquisition time and/or the lengths of the dwell times recorded for the binding events is/are characteristic of a query probe binding to a target analyte and thus provide an indication that the target analyte is immobilized at said discrete location and thus that the target analyte is present in the sample.

Binding of the query probe to the immobilized target analyte and/or and dissociation of the query probe from the immobilized target analyte is/are monitored (e.g., using a light source to excite the fluorescent probe and detecting fluorescence emission from a bound query probe, e.g., using a fluorescence microscope) and/or recorded during a defined time interval (e.g., during the acquisition time). The number of times the query probe binds to the nucleic acid during the acquisition time and/or the length of time the query probe remains bound to the nucleic acid during each binding event and the length of time the query probe remains unbound to the nucleic acid between each binding event (e.g., the “dwell times” in the bound and unbound states, respectively) are determined, e.g., by the use of a computer and software (e.g., to analyze the data using a hidden Markov model and Poisson statistics).

In some embodiments, positive and/or negative control samples are measured (e.g., a control sample known to comprise or not to comprise a target). Fluorescence detected in a negative control sample is “background fluorescence” or “background (fluorescence) intensity” or “baseline”.

In some embodiments, data comprising measurements of fluorescence intensity at the emission wavelength of the query probe are recorded as a function of time. In some embodiments, the number of binding events and the dwell times of binding events (e.g. for each immobilized analyte) are determined from the data (e.g., by determining the number of times and the lengths of time the fluorescence intensity is above a threshold background fluorescence intensity). In some embodiments, transitions (e.g., binding and dissociation of one or more query probes) are counted for each discrete location on the solid support where a target analyte is immobilized. In some embodiments, a threshold number of transitions is used to discriminate the presence of a target analyte at a discrete location on the solid support from background signal, non-target analyte, and/or spurious binding of the query probe.

In some embodiments, a distribution of the number of transitions for each immobilized target is determined—e.g., the number of transitions is counted for each immobilized analyte observed. In some embodiments a histogram is produced. In some embodiments, characteristic parameters of the distribution are determined, e.g., the mean, median, peak, shape, etc. of the distribution are determined. In some embodiments, data and/or parameters (e.g., fluorescence data (e.g., fluorescence data in the time domain), kinetic data, characteristic parameters of the distribution, etc.) are analyzed by algorithms that recognize patterns and regularities in data, e.g., using artificial intelligence, pattern recognition, machine learning, statistical inference, neural nets, etc. In some embodiments, the analysis comprises use of a frequentist analysis and in some embodiments the analysis comprises use of a Bayesian analysis. In some embodiments, pattern recognition systems are trained using known “training” data (e.g., using supervised learning) and in some embodiments algorithms are used to discover previously unknown patterns (e.g., unsupervised learning). See, e.g., Duda, et al. (2001) Pattern classification (2nd edition), Wiley, New York; Bishop (2006) Pattern Recognition and Machine Learning, Springer.

Pattern recognition (e.g., using training sets, supervised learning, unsupervised learning, and analysis of unknown samples) associates identified patterns with analytes such that particular patterns provide a “fingerprint” of particular analytes that find use in detection, quantification, and identification of analytes.

In some embodiments, the distribution produced from a target analyte is significantly different than a distribution produced from a non-target analyte or the distribution produced in the absence of a target analyte. In some embodiments, a mean number of transitions is determined for the plurality of immobilized target analytes. In some embodiments, the mean number of transitions observed for a sample comprising a target analyte is approximately linearly related as a function of time and has a positive slope (e.g., the mean number of transitions increases approximately linearly as a function of time).

In some embodiments, the data are treated using statistics (e.g., Poisson statistics) to determine the probability of a transition occurring as a function of time at each discrete location on the solid support. In some particular embodiments, a relatively constant probability of a transition event occurring as a function of time at a discrete location on the solid support indicates the presence of a target analyte at said discrete location on the solid support. In some embodiments, a correlation coefficient relating event number and elapsed time is calculated from the probability of a transition event occurring as a function of time at a discrete location on the solid support. In some embodiments, a correlation coefficient relating event number and elapsed time greater than 0.95 when calculated from the probability of a transition event occurring as a function of time at a discrete location on the solid support indicates the presence of a target analyte at said discrete location on the solid support.

In some embodiments, dwell times of bound query probe (τₒₙ) and unbound query probe (τ_{off}) are used to identify the presence of a target analyte in a sample and/or to distinguish a sample comprising a target analyte from a sample comprising a non-target analyte and/or not comprising the target analyte. For example, the τₒₙ for a target analyte is greater than the τₒₙ for a non-target analyte; and, the τ_{off} for a target analyte is smaller than the τₒₙ for a non-target analyte. In some embodiments, measuring τₒₙ and τ_{off} for a negative control and for a sample indicates the presence or absence of the target analyte in the sample. In some embodiments, a plurality of τₒₙ and τ_{off} values is determined for each of a plurality of spots imaged on a solid support, e.g., for a control (e.g., positive and/or negative control) and a sample suspected of comprising a target analyte. In some embodiments, a mean τₒₙ and/or τ_{off} is determined for each of a plurality of spots imaged on a solid support, e.g., for a control (e.g., positive and/or negative control) and a sample suspected of comprising a target analyte. In some embodiments, a plot of τₒₙ versus τ_{off} (e.g., mean τₒₙ and τ_{off}, time-averaged τₒₙ and τ_{off}, etc.) for all imaged spots indicates the presence or absence of the target analyte in the sample.

As described herein, the technology detects analytes by a kinetic detection technology. Accordingly, particular embodiments of the technology are related to detecting an analyte by analyzing the kinetics of the interaction of a query probe with the analyte to be detected. For the interaction of a query probe Q (e.g., at an equilibrium concentration [Q]) with a target analyte T (e.g., at an equilibrium concentration [T]), the kinetic rate constant kₒₙ describes the time-dependent formation of the complex QT comprising the probe Q hybridized to the analyte T. In particular embodiments, while the formation of the QT complex is associated with a second order rate constant that is dependent on the concentration of query probe and has units of M⁻¹ min⁻¹ (or the like), the formation of the QT complex is sufficiently described by a kₒₙ that is a pseudo-first order rate constant associated with the formation of the QT complex. Thus, as used herein, kₒₙ is an apparent (“pseudo”) first-order rate constant.

Likewise, the kinetic rate constant k_{off} describes the time-dependent dissociation of the complex QT into the probe Q and the analyte T. Kinetic rates are typically provided herein in units of min⁻¹ or s⁻¹. The “dwell time” of the query probe Q in the bound state (τₒₙ) is the time interval (e.g., length of time) that the probe Q is hybridized to the analyte T during each instance of query probe Q binding to the analyte T to form the QT complex. The “dwell time” of the query probe Q in the unbound state (τ_{off}) is the time interval (e.g., length of time) that the probe Q is not hybridized to the analyte T between each instance of query probe Q binding to the analyte to form the QT complex (e.g., the time the query probe Q is dissociated from the target analyte T between successive binding events of the query probe Q to the target analyte T). Dwell times may be provided as averages or weighted averages integrating over numerous binding and non-binding events.

Further, in some embodiments, the repeated, stochastic binding of probes (e.g., detectably labeled query probes (e.g., fluorescent probes) to target analytes is modeled as a Poisson process occurring with constant probability per unit time and in which the standard deviation in the number of binding and dissociation events per unit time (N_{b+d}) increases as (N_{b+d})^{1/2}. Thus, the statistical noise becomes a smaller fraction of N_{b+d} as the observation time is increased. Accordingly, the observation is lengthened as needed in some embodiments to achieve discrimination between target and off-target binding. And, as the acquisition time is increased, the signal and background peaks in the N_{b+d} histogram become increasingly separated and the width of the signal distribution increases as the square root of N_{b+d} consistent with kinetic Monte Carlo simulations.

Further, in some embodiments assay conditions are controlled to tune the kinetic behavior to improve discrimination of query probe binding events to the target analyte from background binding. For example, in some embodiments the technology comprises control of assay conditions such as, e.g., using a query probe that is designed to interact weakly with the target analyte (e.g., in the nanomolar affinity range); controlling the temperature such that the query probe interacts weakly with the target analyte; controlling the solution conditions, e.g., ionic strength, ionic composition, addition of chaotropic agents, and addition of competing probes.

Some embodiments provide a method of identifying an analyte by repetitive query probe binding. In some embodiments, methods comprise immobilizing an analyte to a solid support. In some embodiments, the solid support is a surface (e.g., a substantially planar surface, a rounded surface), e.g., a surface in contact with a bulk solution, e.g., a bulk solution comprising analyte. In some embodiments, the solid support is a freely diffusible solid support (e.g., a bead, a colloidal particle, e.g., a colloidal particle having a diameter of approximately 10-1000 nm (e.g., 10, 20, 30, 40, 50, 60, 70, 80, 90, 100, 110, 120, 130, 140, 150, 200, 250, 300, 350, 400, 450, 500, 550, 600, 650, 700, 750, 800, 850, 900, 950, or 1000 nm)), e.g., that freely diffuses within the bulk solution, e.g., a bulk solution comprising the analyte. In some embodiments, immobilizing an analyte to a solid support comprises covalent interaction between the solid support and analyte. In some embodiments, immobilizing an analyte to a solid support comprises non-covalent interaction between the solid support and analyte. In some embodiments, the analyte (e.g., a molecule, e.g., a molecule such as, e.g., a protein, peptide, nucleic acid, small molecule, lipid, metabolite, drug, etc.) is stably immobilized to a surface and methods comprise repetitive (e.g., transient, low-affinity) binding of a query probe to the target analyte. In some embodiments, methods comprise detecting the repetitive (e.g., transient, low-affinity) binding of a query probe to the target analyte. In some embodiments, methods comprise generating a dataset comprising a signal produced from query probe binding to the analyte (e.g., a dataset of query probe signal as a function of time) and information (e.g., coordinates, e.g., x, y coordinates) describing the spatial position on the surface of the query probe binding to the analyte. In some embodiments, the dataset is processed (e.g., manipulated, transformed, visualized, etc.), e.g., to improve the spatial resolution of the query probe binding events. For example, in particular embodiments, the dataset (e.g., comprising query probe signal as a function of time and information (e.g., coordinates, e.g., x, y coordinates) describing the spatial position on the surface of the query probe binding to the analyte) is subjected to processing. In some embodiments, the processing comprises a frame-by-frame subtraction process to generate differential intensity profiles showing query probe binding or dissociation events within each frame of the time series data. Data collected during the development of the technology described herein indicate that the differential intensity profiles have a higher resolution than the query probe binding signal vs. position map. In some embodiments, after determining the spatial position (e.g., x, y coordinates) of each query probe binding and/or dissociation event, a plurality of events is clustered according to spatial position and the kinetics of the events within each cluster are subjected to statistical analysis to determine whether the cluster of events originates from a given target analyte.

For instance, some embodiments of methods for quantifying one or more surface-immobilized or diffusing target analytes comprise one or more steps including, e.g., measuring the signal of one or more transiently binding query probes to the immobilized target analyte(s) with single-molecule sensitivity. In some embodiments, methods comprise tracking (e.g., detecting and/or recording the position of) target analytes independently from query probe binding. In some embodiments, the methods further comprise calculating the time-dependent probe binding signal intensity changes at the surface as a function of position (e.g., x, y position). In some embodiments, calculating the time-dependent query probe binding signal intensity changes at the surface as a function of position (e.g., x, y position) produces a “differential intensity profile” for query probe binding to the analyte. In some embodiments, the methods comprise determining the position (e.g., x, y position) of each query probe binding and dissociation event (“event”) with sub-pixel accuracy from a differential intensity profile. In some embodiments, methods comprise grouping events into local clusters by position (e.g., x, y position) on the surface, e.g., to associate events for a single immobilized target analyte. In some embodiments, the methods comprise calculating kinetic parameters from each local cluster of events to determine whether the cluster originates from a particular analyte, e.g., from transient probe binding to a particular analyte.

Embodiments of methods are not limited in the analyte that is detected. For example, in some embodiments the analyte is polypeptide, e.g., a protein or a peptide. In some embodiments, the target analyte is a nucleic acid. In some embodiments, the target analyte is a small molecule.

In some embodiments, the interaction between the target analyte and the query probe is distinguishably influenced by a covalent modification of the target analyte. For example, in some embodiments, the analyte is a polypeptide comprising a post-translational modification, e.g., a protein or a peptide comprising a post-translational modification. In some embodiments, a post-translational modification of a polypeptide affects the transient binding of a query probe with the analyte, e.g., the query probe signal is a function of the presence or absence of the post-translational modification on the polypeptide. For example, in some embodiments, the analyte is a nucleic acid comprising an epigenetic modification, e.g., a nucleic acid comprising a methylated base.

In some embodiments, the analyte is a nucleic acid comprising a covalent modification to a nucleobase, a ribose, or a deoxyribose moiety of the target analyte.

In some embodiments, a modification of a nucleic acid affects the transient binding of a query probe with the analyte, e.g., the query probe signal is a function of the presence or absence of the modification on the nucleic acid.

In some embodiments, the transient interaction between the post-translational modification and the query probe is mediated by a chemical affinity tag, e.g., a chemical affinity tag comprising a nucleic acid.

In some embodiments, the query probe is a nucleic acid or an aptamer.

In some embodiments, the query probe is a low-affinity antibody, antibody fragment, or nanobody.

In some embodiments, the query probe is a DNA-binding protein, RNA-binding protein, or a DNA-binding ribonucleoprotein complex.

In some embodiments, the positon, e.g., the (x,y) position, of each binding or dissociation event is determined by subjecting the differential intensity profile to centroid determination, least-squares fitting to a Gaussian function, least-square fitting to an airy disk function, least-squares fitting to a polynomial function (e.g., a parabola), or maximum likelihood estimation.

In some embodiments, the capture probe is a high-affinity antibody, antibody fragment, or nanobody. In some embodiments, the capture probe is a nucleic acid. In some embodiments, capture is mediated by a covalent bond cross-linking the target analyte to the surface. In some embodiments, the target analyte is subjected to thermal denaturation in the presence of a carrier prior to surface immobilization. In some embodiments, the analyte is subjected to chemical denaturation in the presence of a carrier prior to surface immobilization, e.g., the analyte is denatured with a denaturant such as urea, formamide, guanidinium chloride, high ionic strength, low ionic strength, high pH, low pH, or sodium dodecyl sulfate (SDS).

### Compositions

Embodiments of the technology relate to compositions for detecting, characterizing, identifying, and/or quantifying analytes. Embodiments of the technology comprise a query probe moiety and a capture probe moiety. In some embodiments, the capture probe moiety comprises a label. In some embodiments, the query probe moiety comprises a label. In some embodiments, the capture probe moiety comprises a first label and the query probe moiety comprises a second label. In some embodiments, the first label and the second label are a FRET pair. In some embodiments, the first label comprises a fluorescent moiety and the second label comprises a quencher moiety. The capture probe moiety and the query probe moiety are capable of associating with an analyte. In some embodiments, the capture probe moiety and the query probe moiety are capable of associating with an analyte simultaneously (e.g., in some embodiments, the capture probe moiety and the query probe moiety associate with an analyte simultaneously).

In particular, in some embodiments, the capture probe moiety associates with (e.g., binds to) the analyte (e.g., a first region of the analyte) with high thermodynamic stability (e.g., to form a complex of the capture probe moiety and analyte with a melting temperature more than 10 degrees C. above the measurement temperature or having an average lifetime more than 10 times longer than the observation period). In some embodiments, the capture probe moiety associates with (e.g., binds to) the analyte (e.g., a first region of the analyte) to form a complex of the capture probe moiety and analyte with a melting temperature more than 5 degrees C. (e.g., more than 5, 6, 7, 8, 9, 10, 11, 12, 13, 14, 15, 16, 17, 18, 19, 20, 21, 22, 23, 24, 25, 26, 27, 28, 29, or 30 degrees C.) above the measurement temperature or having an average lifetime more than 5 times (e.g., more than 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 200, 250, 300, 350, 400, 450, 500, or 1000 times) longer than the observation period.

Further, in some embodiments, the query probe moiety associates with the analyte (e.g., a second region of the analyte) with moderate to low thermodynamic stability (e.g., to form a complex of the query probe moiety and analyte with a melting temperature within 10 degrees C. of the measurement temperature or having an average lifetime no more than ⅕th as long as the observation period). In some embodiments, the query probe moiety associates with the analyte (e.g., a second region of the analyte) to form a complex of the query probe moiety and analyte with a melting temperature within 10 degrees C. (e.g., within 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 3.5, 4.0, 4.5, 5.0, 5.5, 6.0, 6.5, 7.0, 7.5, 8.0, 8.5, 9.0, 9.5, or 10.0 degrees C.) of the measurement temperature or having an average lifetime no more than ⅕th (e.g., no more than 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20 times) as long as the observation period.

Some embodiments optionally comprise a decoy moiety. In some embodiments, the decoy moiety associates with the analyte with moderate to low thermodynamic stability (e.g., to form a complex of the decoy moiety and analyte with a melting temperature within 10 degrees C. of the measurement temperature or having an average lifetime no more than ⅕th as long as the observation period). In some embodiments, the decoy moiety associates with the analyte to form a complex of the query probe moiety and analyte with a melting temperature within 10 degrees C. (e.g., within 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 3.5, 4.0, 4.5, 5.0, 5.5, 6.0, 6.5, 7.0, 7.5, 8.0, 8.5, 9.0, 9.5, or 10.0 degrees C.) of the measurement temperature or having an average lifetime no more than ⅕th (e.g., no more than 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20 times) as long as the observation period. In some embodiments the decoy moiety comprises a label. In some embodiments, the decoy moiety comprises a quencher moiety.

In some embodiments, when both the capture probe and the query probe are associated with the analyte, the composition or system is in “State 1”. In some embodiments, State 1 comprises a label of the capture probe and a label of the query probe that are close in proximity (e.g., the distance between the label of the capture probe and a label of the query probe is less than 15 nm (e.g., less than 15, 14, 13, 12, 11, 10, 9, 8, 7, 6, 5, 4, 3, 2, or 1 nm)). In some embodiments, when the query probe is not associated with the analyte, the composition or system is in “State 2”. In some embodiments, State 2 comprises a label of the capture probe and a label of the query probe that are far apart (e.g., the distance between the label of the capture probe and a label of the query probe is more than 5 to 10 nm (e.g., more than 5, 6, 7, 8, 9, 10, 11, 12, 13, 14, 15, 16, 17, 18, 19, 20 or more nm apart)).

Some embodiments optionally comprise an anchor moiety. In some embodiments, an anchor moiety immobilizes the capture probe moiety (e.g., to a solid support, surface, phase boundary). In some embodiments, when the capture probe moiety is bound to the analyte, the anchor moiety immobilizes the capture probe moiety and the analyte (e.g., to a solid support, surface, phase boundary). In some embodiments, the anchor moiety is attached to a solid support (e.g., a surface, bead, particle, phase boundary, etc.). In some embodiments, the anchor moiety comprises an affinity tag or functional group capable of covalent or non-covalent interaction with a surface or phase boundary (e.g., comprising an affinity tag or functional group). In some embodiments, the surface or phase boundary comprises an affinity tag or functional group capable of binding to a moiety of the anchor group (e.g., comprising an affinity tag or functional group). For instance, in some embodiments the anchor moiety comprises biotin (e.g., for binding to avidin and/or streptavidin), streptavidin (e.g., for binding to biotin), digoxigenin (e.g., for binding to anti-digoxigenin), or anti-digoxigenin (e.g., for binding to digoxigenin). In some embodiments, the anchor moiety comprises a click chemistry moiety (e.g., an azide for reacting with an alkyne, an alkyne for reacting with an azide) for click chemistry-mediated immobilization. In some embodiments, the surface or phase boundary comprises biotin (e.g., for binding to avidin and/or streptavidin), streptavidin (e.g., for binding to biotin), digoxigenin (e.g., for binding to anti-digoxigenin), or anti-digoxigenin (e.g., for binding to digoxigenin). In some embodiments, the surface or phase boundary comprises a click chemistry moiety (e.g., an azide for reacting with an alkyne, an alkyne for reacting with an azide) for click chemistry-mediated immobilization of the anchor moiety to the surface, phase boundary, solid support, bead, etc. In some embodiments, the anchor moiety comprises an antibody or epitope-binding fragment of an antibody. In some embodiments, the anchor moiety comprises an epitope that is specifically recognized by an antibody or epitope-binding antibody fragment and/or is capable of being specifically bound by an antibody or epitope-binding antibody fragment. In some embodiments, the surface or phase boundary comprises an antibody or epitope-binding fragment of an antibody. In some embodiments, the surface or phase boundary comprises an epitope that is specifically recognized by an antibody or epitope-binding antibody fragment and/or is capable of being specifically bound by an antibody or epitope-binding antibody fragment.

The technology encompasses various embodiments in which one or more of a query probe moiety, a capture probe moiety, and/or a decoy moiety is/are attached to each other (e.g., by a linker or spacer) and/or is/are attached to an anchor moiety (e.g., by a linker or spacer).

In some embodiments, exemplary covalent connections comprise a nucleic acid, a polypeptide, and/or an organic moiety. In some embodiments, a “linker” as understood in the art connects one or more components of the technology (e.g., a capture probe moiety, a query probe moiety, an optional decoy moiety, and/or an optional anchor moiety). In some embodiments, a “linker” or “spacer” such as the following connects one or more components of the technology (e.g., a capture probe moiety, a query probe moiety, an optional decoy moiety, and/or an optional anchor moiety), e.g., any molecule containing a chain of atoms, e.g., carbon, nitrogen, oxygen, etc., that connects one or more components of the technology (e.g., a capture probe moiety, a query probe moiety, an optional decoy moiety, and/or an optional anchor moiety). In some embodiments, the linker or spacer has a length of, e.g., 1-250 nm (e.g., approximately 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, or 250 nm). In some embodiments, the linker or spacer comprises an alkane chain, e.g., comprising approximately 5 to 2000 C—C bonds or similar bonds (e.g., C—N, C—O, etc.) In some embodiments, the spacer comprises a polyaldehyde, poly(alkyl methacrylate), poly(ethylene oxide), polyolefin, poly(ω-alkenoic acid ester, etc. In some embodiments, a linker or spacer is a nucleic acid comprising 5-50 nt (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nt).

In some embodiments, the capture probe moiety and query probe moiety are directly or indirectly attached to each other. For instance, in some embodiments, a capture probe moiety is covalently connected (e.g., by one or more covalent bonds) to a query probe moiety. In some embodiments, a capture probe moiety is non-covalently connected (e.g., by one or more non-covalent binding interactions) to a query probe moiety. In some embodiments, a capture probe moiety is connected to a query probe moiety by a mixture of covalent bonds and non-covalent interactions.

In some embodiments, the capture probe moiety and query probe moiety are not (e.g., not directly and not indirectly) attached to each other. Accordingly, in some embodiments the capture probe moiety and query probe moiety move essentially, substantially, effectively independently of each other in solution.

In some embodiments, an optional decoy moiety is directly attached to the query probe moiety (e.g., by a linker or spacer as described herein). In some embodiments, one or more of the query probe moiety, capture probe moiety, and/or optional decoy moiety is/are connected directly to an anchor moiety (e.g., by a spacer or linker as described herein).

In some embodiments, an optional decoy is not (e.g., not directly and not indirectly) attached to the query probe. Accordingly, in some embodiments the decoy moiety and query probe moiety move essentially, substantially, effectively independently of each other in solution.

The technology encompasses various embodiments in which one or more of the query probe moiety, capture probe moiety, and/or decoy moiety is/are attached to each other (e.g., by a linker or spacer) and/or is/are attached to an anchor moiety (e.g., by a linker or spacer). Accordingly, in some embodiments the capture probe moiety is attached to an anchor moiety and the query probe moiety is in solution. In some embodiments, the capture probe moiety is attached to an anchor moiety, a query probe is in solution, and a decoy moiety is in solution. In some embodiments, the capture probe moiety is attached to an anchor moiety, a query probe is in solution, and a decoy moiety is attached to the capture probe moiety. In some embodiments, the capture probe moiety is attached to an anchor probe moiety, a query probe is attached to the capture probe moiety, and a decoy moiety is in solution. In some embodiments, the capture probe moiety is attached to an anchor probe moiety, a query probe is attached to the capture probe moiety, and a decoy moiety is attached to the capture probe moiety.

In some embodiments, the capture probe moiety is attached to an anchor moiety and the query probe moiety is attached to the decoy moiety, but neither the query probe moiety nor the decoy moiety is attached to the capture probe moiety and neither the query probe moiety nor the decoy moiety is attached to the anchor moiety.

In some embodiments, the capture probe moiety is attached to the query probe moiety. In some embodiments, the capture probe moiety is attached to the decoy moiety.

In some embodiments, the decoy moiety is attached to the query probe moiety. In some embodiments, the capture probe moiety, query probe moiety, and decoy moiety are all attached to each other.

In these various embodiments, the various attachments can be direct, indirect, or a combination of direct and indirect. That is, in various embodiments the various attached components are directly or indirectly attached to each other. For instance, in some embodiments, a first component (e.g., a capture probe moiety, a query probe moiety, an optional decoy moiety, an optional anchor moiety) is covalently connected (e.g., by one or more covalent bonds) to a second component (e.g., a capture probe moiety, a query probe moiety, an optional decoy moiety, an optional anchor moiety). In some embodiments, a first component (e.g., a capture probe moiety, a query probe moiety, an optional decoy moiety, an optional anchor moiety) is non-covalently connected (e.g., by one or more non-covalent binding interactions) to a second component (e.g., a capture probe moiety, a query probe moiety, an optional decoy moiety, an optional anchor moiety). In some embodiments, a first component (e.g., a capture probe moiety, a query probe moiety, an optional decoy moiety, an optional anchor moiety) is connected to a second component (e.g., a capture probe moiety, a query probe moiety, an optional decoy moiety, an optional anchor moiety) by a mixture of covalent bonds and non-covalent binding interactions.

In some embodiments, compositions comprise an imaging buffer. In some embodiments, an imaging buffer comprises a pH buffer, one or more salts, and water. In some embodiments, an imaging buffer comprises an ionic chaotropic agents. In some embodiments, an imaging buffer comprises a nonionic chaotropic agent. In some embodiments, an imaging buffer comprises guanidine hydrochloride, formamide, or urea. In some embodiments, an imaging buffer comprises approximately 10% formamide (e.g., approximately 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, or 20.0% formamide).

### FRET Pairs and Flour-Quencher Pairs

Förster or fluorescence resonance energy transfer (FRET) is a physical phenomenon in which a donor fluorophore in its excited state non-radiatively transfers its excitation energy to a neighboring acceptor fluorophore, thereby causing the acceptor to emit its characteristic fluorescence.

“FET”, as used herein, refers to “Fluorescence Energy Transfer.” “FRET”, as used herein, refers to “Fluorescence Resonance Energy Transfer.” These terms are used herein to refer to both radiative and non-radiative energy transfer processes. For example, processes in which a photon is emitted and those involving long-range electron transfer are included within these terms. In some embodiments, both of these phenomena are subsumed under the general term “donor-acceptor energy transfer.”

As used herein, a “FRET pair” refers to a pair of fluorescent labels wherein when the first label is excited at its excitation wavelength, it emits at the excitation wavelength of the second label, thereby causing, if the two labels are in close proximity, the second label to emit at its emission wavelength. Thus, a FRET pair, when excited at the excitation wavelength of the first label, emits at the emission wavelength of the second label. FRET is well known to those in the art. See, e.g., Ishikawa-Ankerhold et al. (2012) “Advanced fluorescence microscopy techniques—FRAP, FLIP, FLAP, FRET and FLIM” Molecules 17(4): 4047-132; Shrestha et al. (2015) “Understanding FRET as a research tool for cellular studies” Int J Mol Sci 16(4): 6718-56; Bajar et al. (2016) “A Guide to Fluorescent Protein FRET Pairs” Sensors (Basel) 16(9); and Bunt and Wouters (2017) “FRET from single to multiplexed signaling events” Biophys Rev 9(2): 119-129, each of which is incorporated herein by reference in its entirety.

Some embodiments relate to use of a FRET pair. For example, in some embodiments a capture probe moiety comprises a first member of a FRET pair (e.g., a first fluorescent dye) and a query probe moiety comprises a second member of a FRET pair (e.g., a second fluorescent dye).

In some embodiments, the fluorophore is an organic fluorophore. In some embodiments, the organic fluorophore comprises, for example, a charged (e.g., ionic) molecule (e.g., sulfonate or ammonium groups), uncharged (e.g., neutral) molecule, saturated molecule, unsaturated molecule, cyclic molecule, bicyclic molecule, tricyclic molecule, polycyclic molecule, acyclic molecule, aromatic molecule, and/or heterocyclic molecule (e.g., by being ring-substituted by one or more heteroatoms selected from, for example, nitrogen, oxygen and sulfur). In the particular case of unsaturated fluorophores, the fluorophore contains one, two, three, or more carbon-carbon and/or carbon-nitrogen double and/or triple bonds. In a particular embodiment, the fluorophore contains at least two (e.g., two, three, four, five, or more) conjugated double bonds aside from any aromatic group that may be in the fluorophore. In other embodiments, the fluorophore is a fused polycyclic aromatic hydrocarbon (PAH) containing at least two, three, four, five, or six rings (e.g., naphthalene, pyrene, anthracene, chrysene, triphenylene, tetracene, azulene, and phenanthrene) wherein the PAH can be optionally ring-substituted or derivatized by one, two, three or more heteroatoms or heteroatom-containing groups.

The fluorophores considered herein can absorb and emit light of any wavelengths. However, in different embodiments, it may be desired to select a fluorophore with particular absorption and emission characteristics. For example, in different embodiments, the fluorophore preferably absorbs at nanometer (nm) wavelengths of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, or 800 nm, or within a range bounded by any two of the foregoing values. In different embodiments, the fluorophore preferably emits at any of the foregoing wavelengths, or within a range bounded by any two of the foregoing values, wherein it is understood that a fluorophore generally emits at a higher wavelength than the absorbed wavelength. The impinging electromagnetic radiation (e.g., which is absorbed by the fluorophore) can be in a dispersed form, or alternatively, in a focused form, such as a laser. Moreover, the absorbed or emitted radiation can be in the form of, for example, far infrared, infrared, far red, visible, near-ultraviolet, or ultraviolet. When two or more fluorophores are used (e.g., attached to a biomolecule, as in FRET and smFRET methods), one of the fluorophores functions as a donor fluorophore and the other functions as an acceptor fluorophore.

The technology is not limited in the type, structure, or composition of the fluorescent moiety. Non-limiting examples of fluorescent moieties include dyes that can be synthesized or obtained commercially (e.g., Operon Biotechnologies, Huntsville, Ala.). A large number of dyes (greater than 50) are available for application in fluorescence excitation applications. These dyes include those from the fluorescein, rhodamine, AlexaFluor, Bodipy, Coumarin, and Cyanine dye families. Specific examples of fluorophores include, but are not limited to, FAM, TET, HEX, Cy3, TMR, ROX, VIC (e.g., from Life Technologies), Texas red, LC red 640, Cy5, and LC red 705. In some embodiments, dyes with emission maxima from 410 nm (e.g., Cascade Blue) to 775 nm (e.g., Alexa Fluor 750) are available and can be used. Of course, one of ordinary skill in the art will recognize that dyes having emission maxima outside these ranges may be used as well. In some cases, dyes ranging between 500 nm to 700 nm have the advantage of being in the visible spectrum and can be detected using existing photomultiplier tubes. In some embodiments, the broad range of available dyes allows selection of dye sets that have emission wavelengths that are spread across the detection range. Detection systems capable of distinguishing many dyes are known in the art.

Accordingly, in some embodiments, the organic fluorophore is a xanthene derivative (e.g., fluorescein, rhodamine, Oregon green, eosin, and Texas Red), cyanine or its derivatives or subclasses (e.g., streptocyanines, hemicyanines, closed chain cyanines, phycocyanins, allophycocyanins, indocarbocyanines, oxacarbocyanines, thiacarbocyanines, merocyanins, and phthalocyanines), naphthalene derivatives (e.g., dansyl and prodan derivatives), coumarin and its derivatives, oxadiazole and its derivatives (e.g., pyridyloxazoles, nitrobenzoxadiazoles, and benzoxadiazoles), pyrene and its derivatives, oxazine and its derivatives (e.g., Nile Red, Nile Blue, and cresyl violet), acridine derivatives (e.g., proflavin, acridine orange, and acridine yellow), arylmethine derivatives (e.g., auramine, crystal violet, and malachite green), and tetrapyrrole derivatives (e.g., porphyrins and bilirubins). Some particular families of dyes considered herein are the CY family of dyes (e.g., Cy2, Cy3, Cy3B, Cy3.5, Cy5, Cy5.5, and Cy7), the ALEXA family of dyes (e.g., ALEXA Fluor 350, 405, 430, 488, 500, 514, 532, 546, 555, 568, 594, 610, 633, 647, 660, 680, 700, 750, and 790), the ATTO family of dyes (e.g., ATTO 390, 425, 465, 488, 495, 520, 532, 550, 565, 590, 594, 601, 615, 619, 629, 635, 645, 663, 680, 700, 729, and 740), and the DY family of dyes (e.g., DY 530, 547, 548, 549, 550, 554, 556, 560, 590, 610, 615, 630, 631, 631, 632, 633, 634, 635, 636, 647, 648, 649, 650, 651, 652, 675, 676, 677, 680, 681, 682, 700, 701, 730, 731, 732, 734, 750, 751, 752, 776, 780, 781, 782, and 831). The ATTO dyes, in particular, can have several structural motifs, including, coumarin-based, rhodamine-based, carbopyronin-based, and oxazine-based structural motifs.

In some embodiments, fluorescent dyes include, without limitation, dyes such as d-Rhodamine acceptor dyes including Cy5, dichloro[R110], dichloro[R6G], dichloro[TAMRA], dichloro[ROX] or the like, fluorescein donor dye including fluorescein, 6-FAM, or the like; Acridine including Acridine orange, Acridine yellow, Proflavin, pH 7, or the like; Aromatic Hydrocarbon including 2-Methylbenzoxazole, Ethyl p-dimethylaminobenzoate, Phenol, Pyrrole, benzene, toluene, or the like; Arylmethine Dyes including Auramine O, Crystal violet, H₂O, Crystal violet, glycerol, Malachite Green or the like; Coumarin dyes including 7-Methoxycoumarin-4-acetic acid, Coumarin 1, Coumarin 30, Coumarin 314, Coumarin 343, Coumarin 6 or the like; Cyanine Dye including 1,1′-diethyl-2,2′-cyanine iodide, Cryptocyanine, Indocarbocyanine (C3)dye, Indodicarbocyanine (C5)dye, Indotricarbocyanine (C7)dye, Oxacarbocyanine (C3)dye, Oxadicarbocyanine (C5)dye, Oxatricarbocyanine (C7)dye, Pinacyanol iodide, Stains all, Thiacarbocyanine (C3)dye, ethanol, Thiacarbocyanine (C3)dye, n-propanol, Thiadicarbocyanine (C5)dye, Thiatricarbocyanine (C7)dye, or the like; Dipyrrin dyes including N,N′-Difluoroboryl-1,9-dimethyl-5-(4-iodophenyl)-dipyrrin, N,N′-Difluoroboryl-1,9-dimethyl-5-[(4-(2-trimethylsilylethynyl), N,N′-Difluoroboryl-1,9-dimethyl-5-phenydipyrrin, or the like; Merocyanines including 4-(dicyanomethylene)-2-methyl-6-(p-dimethylaminostyryl)-4H-pyran (DCM), acetonitrile, 4-(dicyanomethylene)-2-methyl-6-(p-dimethylaminostyryl)-4H-pyran (DCM), methanol, 4-Dimethylamino-4′-nitrostilbene, Merocyanine 540, or the like; Miscellaneous Dye including 4′,6-Diamidino-2-phenylindole (DAPI), 4,6′-Diamidino-2-phenylindole (DAPI), dimethylsulfoxide, 7-Benzylamino-4-nitrobenz-2-oxa-1,3-diazole, Dansyl glycine, H₂O, Dansyl glycine, dioxane, Hoechst 33258, DMF, Hoechst 33258, H₂O. Lucifer yellow CH, Piroxicam, Quinine sulfate, 0.05 M H₂SO₄, Quinine sulfate, 0.5 M H₂SO₄, Squarylium dye III, or the like; Oligophenylenes including 2,5-Diphenyloxazole (PPO), Biphenyl, POPOP, p-Quaterphenyl, p-Terphenyl, or the like; Oxazines including Cresyl violet perchlorate, Nile Blue, methanol, Nile Red, Nile blue, ethanol, Oxazine 1, Oxazine 170, or the like; Polycyclic Aromatic Hydrocarbons including 9,10-Bis(phenylethynyl)anthracene, 9,10-Diphenylanthracene. Anthracene, Naphthalene, Perylene, Pyrene, or the like; polyene/polyynes including 1,2-diphenylacetylene, 1,4-diphenylbutadiene, 1,4-diphenylbutadiyne, 1,6-Diphenylhexatriene, Beta-carotene, Stilbene, or the like; Redox-active Chromophores including Anthraquinone, Azobenzene, Benzoquinone, Ferrocene, Riboflavin, Tris(2,2′-bipyridypruthenium(II), Tetrapyrrole, Bilirubin, Chlorophyll a, diethyl ether, Chlorophyll a, methanol, Chlorophyll b. Diprotonated-tetraphenylporphyrin, Hematin, Magnesium octaethylporphyrin, Magnesium octaethylporphyrin (MgOEP), Magnesium phthalocyanine (MgPc), PrOH, Magnesium phthalocyanine (MgPc), pyridine, Magnesium tetramesitylporphyrin (MgTMP), Magnesium tetraphenylporphyrin (MgTPP), Octaethylporphyrin, Phthalocyanine (Pc), Porphin, Rox, TAMRA, Tetra-t-butylazaporphine, Tetra-t-butylnaphthalocyanine, Tetrakis(2,6-dichlorophenyl)porphyrin, Tetrakis(o-aminophenyl)porphyrin, Tetramesitylporphyrin (TMP), Tetraphenylporphyrin (TPP), Vitamin B12, Zinc octaethylporphyrin (ZnOEP), Zinc phthalocyanine (ZnPc), pyridine, Zinc tetramesitylporphyrin (ZnTIP), Zinc tetramesitylporphyrin radical cation, Zinc tetraphenylporphyrin (ZnTPP), or the like; Xanthenes including Eosin Y, Fluorescein, basic ethanol, Fluorescein, ethanol, Rhodamine 123, Rhodamine 6G, Rhodamine B, Rose bengal. Sulforhodamine 101, or the like.

In another embodiment, the fluorophore has an inorganic composition. For example, the fluorophore can include a fluorescent transition metal or rare earth (e.g., lanthanide) metal species or particle (e.g., nanoparticle or microparticle). The transition metal or rare earth metal species can be, for example, a metal-ligand complex. The ligand can be any suitable ligand, such as, for example, acetylacetonate, a Schiff base (e.g., salen), amine, phosphine, thiol, phenanthroline, bipyridine, or phenolate-based ligand. By “transition metal” is meant any of the metals in Groups IB to VIIIB of the Periodic Table. By “rare earth metal” is meant any of the lanthanides and actinides with atomic numbers of, respectively, 57-71 and 90-103. Some particular rare earth metals considered herein include lanthanum, cerium, praseodymium, neodymium, promethium, samarium, europium, gadolinium, terbium, dysprosium, holmium, erbium, thulium, ytterbium, lutetium, and hafnium, and combinations thereof.

In one embodiment, a fluorescent nanoparticle contains a non-fluorescent matrix (e.g., a polymer, ceramic, or metal) into which is included an organic or inorganic fluorescent species (e.g., within or on the surface of the nanoparticle). In particular embodiments, the particle includes an organic polymer (e.g., polystyrene) or inorganic polymer (e.g., silica or siloxane-based). In another embodiment, the particle includes a metal, e.g., nanoparticles based on copper, silver, gold, palladium, or platinum, or a combination thereof. In another embodiment, the fluorescent particle has a semiconductor (e.g., quantum dot) composition. The quantum dot particle typically includes a sulfide, selenide, telluride, nitride, phosphide, arsenide, and/or antimonide of a Group IB element (e.g., Cu or Ag), Group II element (e.g., Zn or Cd), or Group III element (e.g., B, Al, Ga, or In), or combination of these elements. Furthermore, the quantum dot can be essentially homogeneous in structure, or alternatively, layered (e.g., a core-shell quantum dot). The core and shell of such a quantum dot can, independently, be composed of any of the semiconductor compositions described above (e.g., “core:shell” compositions of the type ZnS:CdSe, ZnSe:CdSe, ZnS:CdS, ZnSe:CdS, CdS:ZnSe, CdSe:ZnSe, CdS:ZnS, or CdSe:ZnS). In some embodiments, the quantum dot further includes a dopant fluorescent species, such as any of the rare earth metals or organic fluorescent species described above, either by being incorporated into the core or shell of the quantum dot or by being adsorbed or linked to the surface or passivation shell of the quantum dot.

In different embodiments, the particle (particularly, a quantum dot) possesses a size (e.g., diameter) of, for example, 1, 2, 5, 10, 15, 20, 30, 40, 50, 60, 70, 80, 90, 100, 110, or 120 nm, or a size within a range bounded by any two of the foregoing values. A metal particle that is not a quantum dot may be any of the foregoing sizes, as well as significantly larger sizes, e.g., about, at least, or no more than 150, 200, 250, 300, 350, 400, or 500 nm or a size within a range bounded by any two of the foregoing values. A polymer particle can be any of the foregoing sizes, as well as significantly larger sizes. e.g., about, at least, or no more than 600, 700, 800, 900, 1000, 2000, 3000, 4000, or 5000 nm, or a size within a range bounded by any two of the foregoing values.

Some embodiments relate to use of a fluorescent dye and a quencher moiety. For example, in some embodiments a capture probe moiety comprises a fluorescent dye and a query probe moiety comprises a quencher moiety; in some embodiments a capture probe moiety comprises a quencher moiety and a query probe moiety comprises a fluorescent dye. As used herein, “quenching group” or “quenching moiety” and similar terms refers to any fluorescence-modifying group that can attenuate, at least partly, the energy (e.g., light) emitted by a fluorescent dye. This attenuation is referred to herein as “quenching”. Hence, irradiation of the fluorescent dye in the presence of the quenching group leads to an emission signal from the fluorescent dye that is less intense than expected, or even completely absent. Quenching typically occurs through energy transfer between the fluorescent dye and the quenching group.

Further, the technology is not limited in the type, structure, or composition of the quenching moiety. Exemplary quenching moieties include a Black Hole Quencher, an Iowa Black Quencher, and derivatives, modifications thereof, and related moieties. Exemplary quenching moieties include BHQ-0, BHQ-1, BHQ-2, and BHQ-3.

In some embodiments, fluorescent dye-quencher pairs include, e.g., DLO-FB1 (5′-FAM/3′-BHQ-1) DLO-TEB1 (5′-TET/3′-BHQ-1), DLO-JB1 (5′-JOE/3′-BHQ-1), DLO-1-HB1 (5′-HEX/3′-BHQ-1), DLO-C3B2 (5′-Cy3/3′-BHQ-2), DLO-TAB2 (5′-TAMRA/3′-BHQ-2), DLO-RB2 (5′-ROX/3′-BHQ-2), DLO-C5B3 (5′-Cy5/3′-BHQ-3), DLO-C55B3 (5′-Cy5.5/3′-BHQ-3), MBO-FB1(5′-FAM/3′-BHQ-1), MBO-TEB1 (5′-TET/3′-BHQ-1), MBO-JB1 (5-JOE/3′-BHQ-1), MBO-HB1 (5′-HEX/3′-BHQ-1), MBO-C3B2 (5′-Cy3/3′-BHQ-2), MBO-TAB2 (5′-TAMRA/3′-BHQ-2), MBO-RB2 (5′-ROX/3′-BHQ-2); MBO-C5B3 (5′-Cy5/3′-BHQ-3), MBO-C55B3 (5′-Cy5.5/3′-BHQ-3) or similar FRET pairs available from Biosearch Technologies, Inc. of Novato, Calif. See, e.g., U.S. Pat. App. Pub. No. US20100317005 incorporated herein by reference.

### Decoys

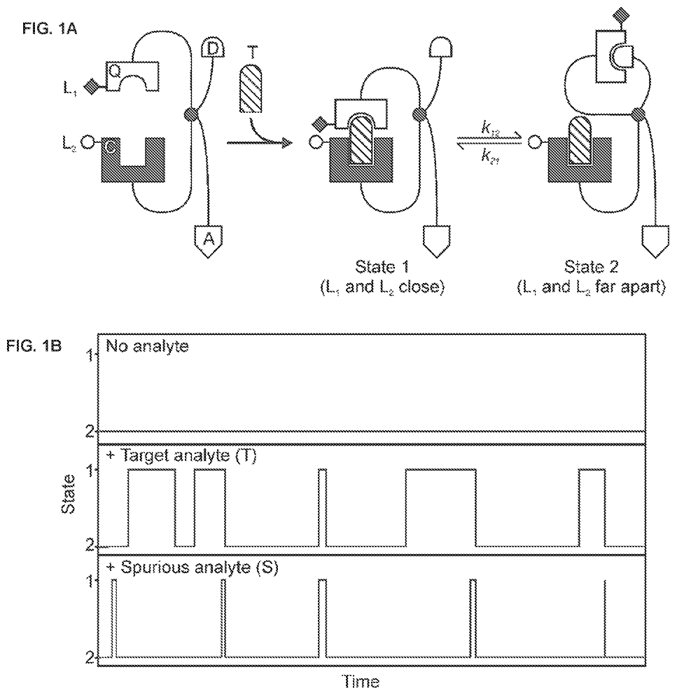

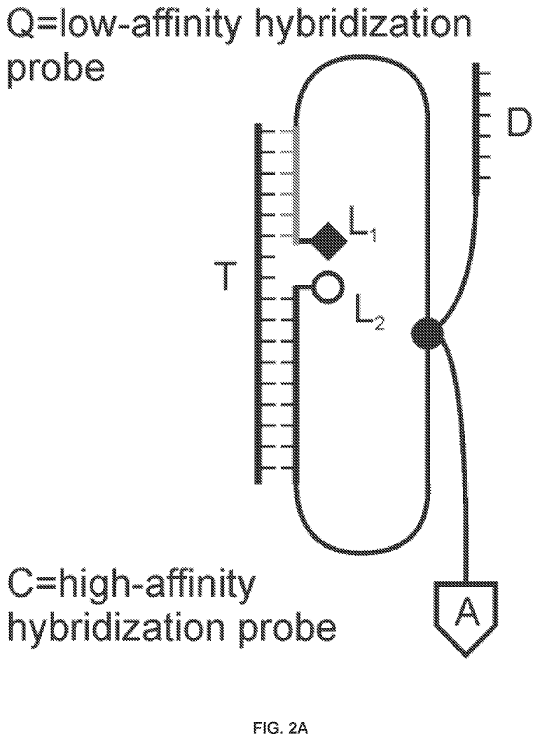

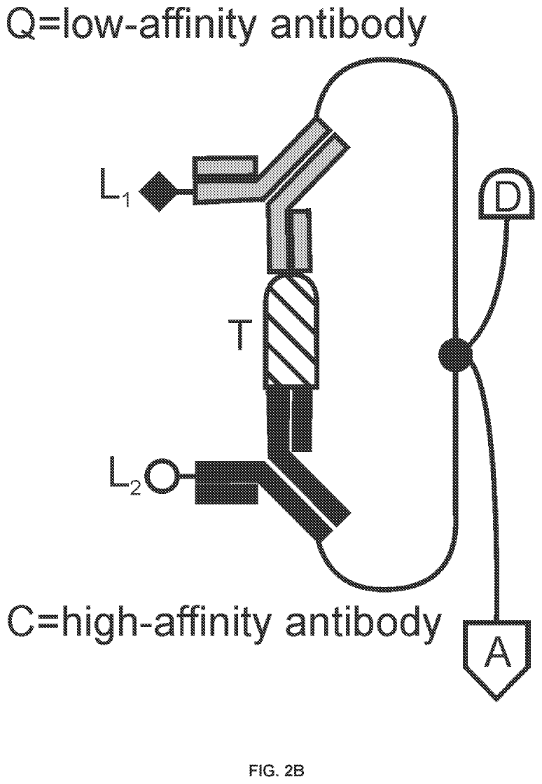

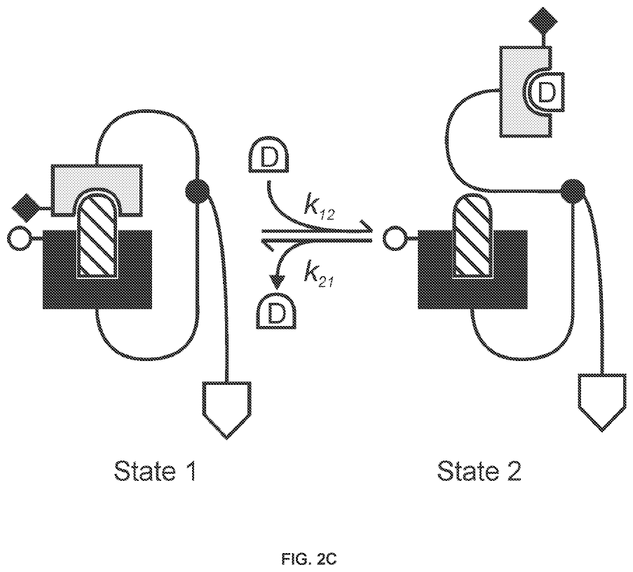

Some embodiments comprise an optional decoy moiety. The decoy moiety binds weakly to the query probe moiety, thus competing with the analyte for binding to the query probe moiety. Accordingly, the kinetics of the interaction of the query probe moiety and the analyte in the presence of the decoy moiety are different than the kinetics of the interaction of the query probe moiety and the analyte in the absence of the decoy moiety. In particular, the decoy moiety prolongs the lifetime of State 2 (query probe dissociated from analyte) relative to State 1 (query probe associated with analyte). As described herein, the decoy moiety is, in some embodiments, connected to the query probe and/or other components of the technology covalently or non-covalently (as depicted in FIG. 1a). In some embodiments, the decoy moiety is introduced “in trans” (e.g., in solution) (as depicted in FIG. 2c). In some embodiments, the decoy moiety is similar to the analyte, e.g., a protein having a similar structure, primary sequence, epitope, affinity for the query probe, etc, as the analyte; a nucleic acid having a similar structure, primary sequence, epitope, affinity for the query probe, etc, as the analyte; a small molecule having a similar structure, primary sequence, epitope, affinity for the query probe, etc. as the analyte. For instance, in some embodiments, the decoy is a polypeptide having a primary amino acid sequence that is 90% or more identical (e.g., 91, 92, 93, 94, 95, 96, 97, 98, 99, 99.2, 99.5, 99.6, 99.7, 99.8, or 99.9% identical) to the primary amino acid sequence of the analyte. In some embodiments, the decoy is a nucleic acid having a primary nucleotide sequence that is 90% or more identical (e.g., 91, 92, 93, 94, 95, 96, 97, 98, 99, 99.2, 99.5, 99.6, 99.7, 99.8, or 99.9% identical) to the primary nucleotide sequence of the analyte. In some embodiments, the decoy is a polypeptide having a primary amino acid sequence comprising one or more substitutions (e.g., 1, 2, 3, 4, 5, 6, 7, 8, 9, or 10 substitutions) with respect to the primary amino acid sequence of the analyte. In some embodiments, the decoy is a nucleic acid having a primary nucleotide sequence comprising one or more mutations (e.g., 1, 2, 3, 4, 5, 6, 7, 8, 9, or 10 mutations) with respect to the primary nucleotide sequence of the analyte. In some embodiments, the decoy is a nucleic acid and/or polypeptide having a primary nucleotide and/or amino acid sequence comprising one or more modifications (e.g., 1, 2, 3, 4, 5, 6, 7, 8, 9, or 10 modifications) with respect to the primary nucleotide and/or amino acid sequence of the analyte. In some embodiments, the decoy is a molecule having a structure comprising one or more different substituents (e.g., “R” groups) (e.g., 1, 2, 3, 4, 5, 6, 7, 8, 9, or 10 different substituents (e.g., “R” groups) than the substituents (e.g., “R” groups) of the structure of the analyte.

### Methods

Embodiments of methods are provided in accordance with the technology. For example, in some embodiments the technology comprises providing a capture probe moiety (e.g., comprising a fluorescent moiety and/or a quencher moiety) and a query probe moiety (e.g., comprising a fluorescent moiety and/or a quencher moiety). In some embodiments, the technology comprises providing a capture probe moiety (e.g., comprising a first fluorescent moiety) and a query probe moiety (e.g., comprising a second fluorescent moiety). In some embodiments, the first and second fluorescent moieties are a FRET pair. In some embodiments, a fluorescent moiety and a quencher moiety are a fluor-quencher pair. Some embodiments further provide an optional decoy moiety as described herein and/or an optional anchor moiety as described herein.

Some embodiments comprise providing a surface, substrate, and/or phase boundary that interacts directly (e.g., by one or more covalent bonds) with an anchor moiety or than interacts non-covalently (e.g., by one or more non-covalent binding interactions) to an anchor moiety. Accordingly, some embodiments comprise immobilizing an anchor moiety, e.g., to a surface, substrate, bead, solid support, and/or phase boundary). For example, in some embodiments the technology comprises providing a surface, substrate, bead, solid support, and/or phase boundary and contacting an anchor moiety to the surface, substrate, bead, solid support, and/or phase boundary. In some embodiments, the technology comprises reacting the anchor moiety with the surface, substrate, bead, solid support, and/or phase boundary to attach the anchor moiety to the surface, substrate, bead, solid support, and/or phase boundary by a covalent bond (e.g., by a chemical reaction that creates a covalent bond between a reactive group of the anchor moiety and a reactive group of the surface, substrate, bead, solid support, and/or phase boundary (e.g., by a click chemistry reaction)). In some embodiments, the technology comprises contacting an anchor moiety to the surface, substrate, bead, solid support, and/or phase boundary to form a thermodynamically stable non-covalent binding interaction between the anchor moiety and the surface, substrate, bead, solid support, and/or phase boundary.

In some embodiments, the anchor moiety is attached to a capture probe. In some embodiments, the anchor moiety is attached to one or more of a capture probe, a query probe, and or an optional decoy.

In some embodiments, the technology further comprises contacting the capture probe with a sample. In some embodiments, the sample is suspected of comprising an analyte. In some embodiments, the sample comprises an analyte.

In some embodiments, the technology comprises providing an imaging buffer. In some embodiments, an imaging buffer comprises a pH buffer, one or more salts, and water. In some embodiments, an imaging buffer comprises an ionic chaotropic agents. In some embodiments, an imaging buffer comprises a nonionic chaotropic agent. In some embodiments, an imaging buffer comprises guanidine hydrochloride, formamide, or urea. In some embodiments, an imaging buffer comprises approximately 10% formamide (e.g., approximately 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, or 20.0% formamide). In some embodiments, methods comprise detecting binding of a query probe to a target in an imaging buffer.

In some embodiments, the technology comprises contacting an analyte to the capture probe. In particular, in some embodiments, the capture probe moiety associates with (e.g., binds to) the analyte (e.g., a first region of the analyte) with high thermodynamic stability. For example, some embodiments comprise forming a complex comprising the capture probe and analyte with a melting temperature more than 10 degrees C. above the measurement temperature or having an average lifetime more than 10 times longer than the observation period. In some embodiments, the technology comprises forming a complex comprising the capture probe and analyte with a melting temperature more than 5 degrees C. (e.g., more than 5, 6, 7, 8, 9, 10, 11, 12, 13, 14, 15, 16, 17, 18, 19, 20, 21, 22, 23, 24, 25, 26, 27, 28, 29, or 30 degrees C.) above the measurement temperature or having an average lifetime more than 5 times (e.g., more than 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 200, 250, 300, 350, 400, 450, 500, or 1000 times) longer than the observation period.

Then, some embodiments comprise providing and/or contacting the analyte with a query probe. In some embodiments, the technology comprises contacting the analyte with a query probe that associates with the analyte (e.g., a second region of the analyte) with moderate to low thermodynamic stability. Some embodiments comprise forming a complex comprising the query probe and analyte having a melting temperature within 10 degrees C. of the measurement temperature or having an average lifetime no more than ⅕th as long as the observation period. Some embodiments comprise forming a complex comprising the query probe moiety and analyte having a melting temperature within 10 degrees C. (e.g., within 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 3.5, 4.0, 4.5, 5.0, 5.5, 6.0, 6.5, 7.0, 7.5, 8.0, 8.5, 9.0, 9.5, or 10.0 degrees C.) of the measurement temperature or having an average lifetime no more than ⅕th (e.g., no more than 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20 times) as long as the observation period.

Furthermore, some embodiments comprise providing a decoy moiety. Some embodiments comprise contacting the decoy moiety with a query probe. In some embodiments, the technology comprises contacting the decoy moiety with a query probe that associates with the decoy moiety with moderate to low thermodynamic stability.

Some embodiments comprise forming a complex comprising the query probe and decoy moiety having a melting temperature within 10 degrees C. of the measurement temperature or having an average lifetime no more than ⅕th as long as the observation period. Some embodiments comprise forming a complex comprising the query probe and decoy moiety having a melting temperature within 10 degrees C. (e.g., within 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 3.5, 4.0, 4.5, 5.0, 5.5, 6.0, 6.5, 7.0, 7.5, 8.0, 8.5, 9.0, 9.5, or 10.0 degrees C.) of the measurement temperature or having an average lifetime no more than ⅕th (e.g., no more than 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20 times) as long as the observation period.

Embodiments of the technology comprise forming a thermodynamically stable complex comprising an analyte and capture moiety and contacting the analyte with a query probe moiety that associates with the analyte (e.g., a second region of the analyte) with moderate to low thermodynamic stability. Thus, the technology comprises forming a transient complex comprising the analyte, capture probe moiety, and query probe moiety. In other words, the technology comprises forming a complex comprising the analyte and capture probe moiety that transiently associates with the query probe moiety.

In some embodiments, upon formation of the transient complex (comprising the analyte, capture probe moiety, and query probe moiety), a first fluorescent moiety of the capture probe moiety and a second fluorescent moiety of the query probe moiety are brought in close proximity to effect FRET between the first fluorescent moiety and the second fluorescent moiety. In some embodiments, upon formation of the transient complex (comprising the analyte, capture probe moiety, and query probe moiety), a fluorescent moiety of the capture probe moiety and a quencher moiety of the query probe moiety are brought in close proximity to quench fluorescence of the fluorescent moiety.

In some embodiments, upon formation of the transient complex (comprising the analyte, capture probe moiety, and query probe moiety), a quencher moiety of the capture probe moiety and a fluorescent moiety of the query probe moiety are brought in close proximity to quench fluorescence of the fluorescent moiety. Changes in fluorescence (e.g., increase, decrease, and/or change in emission maximum (e.g., increase and/or decrease at an emission wavelength or over a range of emission wavelengths)) indicate changes in the relative position in space of the first and second fluorescent moieties or the fluorescent moiety and quencher moiety. Detecting FRET or quenching indicates transient association of the query probe with the analyte.

Thus in some embodiments, the technology comprises providing an excitation wavelength, e.g., contacting a fluorescent moiety (e.g., a first fluorescent moiety) of the capture probe or query probe with a photon having a wavelength that excites the fluorescent moiety (e.g., first fluorescent moiety). Some embodiments comprise exciting the fluorescent moiety of the capture probe and some embodiments comprise exciting the fluorescent moiety of the query probe. Embodiments comprise detecting emission of a fluorescent moiety (e.g., a second fluorescent moiety) of the capture probe or query probe, e.g., detecting a photon having a wavelength characteristic of the emitting fluorescent moiety (e.g., second fluorescent moiety). In some embodiments, the fluorescent moiety of the query probe is excited by a photon and the fluorescent moiety of the capture probe emits a photon that is detected. In some embodiments, the fluorescent moiety of the capture probe is excited by a photon and the fluorescent moiety of the query probe emits a photon that is detected. Accordingly, some embodiments comprise providing an excitation wavelength (a first wavelength) and detecting an emission wavelength (a second wavelength). Some embodiments comprise recording the emission wavelength as a function of time, e.g., to provide a time-dependent signal of the emission wavelength (e.g., to provide a time-dependent signal of the intensity of the emission wavelength).

In some embodiments, the fluorescence emission signal is produced when the fluorescent moiety of the query probe is close to the fluorescent moiety of the capture probe (e.g., the distance between the fluorescent moiety of the capture probe and the fluorescent moiety of the query probe is less than 15 nm (e.g., less than 15, 14, 13, 12, 11, 10, 9, 8, 7, 6, 5, 4, 3, 2, or 1 nm)). When query probes are not associated with the analyte (e.g., when the distance between the fluorescent moiety of the capture probe and the fluorescent moiety of the query probe is more than 5 to 10 nm (e.g., more than 5, 6, 7, 8, 9, 10, 11, 12, 13, 14, 15, 16, 17, 18, 19, 20 or more nm apart)), a FRET signal is not observed.

In some embodiments, the technology is similar except that a fluorescent moiety and a quencher are used such that the fluorescent moiety has a high fluorescence intensity at its emission wavelength when the fluorescent moiety and quenching moiety are far apart and the fluorescent moiety has a low fluorescence intensity at its emission wavelength when the fluorescent moiety and quenching moiety are close together.

Thus, in some embodiments, the technology comprises monitoring fluorescence emission at a number of discrete locations (e.g., on a solid support where the analytes are immobilized), e.g., at a number of fluorescent “spots” that blink, e.g., that can be in “on” and “off states. The presence of fluorescence emission (spot is “on”) and absence of fluorescence emission (spot is “off”) at each discrete location (e.g., at each “spot” on the solid support) are recorded. Each spot “blinks”—e.g., a spot alternates between “on” and “off” states, respectively, as a query probe binds to the immobilized target analyte at that spot and as the query probe dissociates from the immobilized target analyte at that spot (in the case of a FRET method) and a spot alternates between “off” and “on” states, respectively, as a query probe binds to the immobilized target analyte at that spot and as the query probe dissociates from the immobilized target analyte at that spot (in the case of a quenching method).

Thus, detecting fluorescence emission at the emission wavelength of the fluorescent label indicates that the query probe is not bound to an immobilized analyte in a quenching method. Alternatively, detecting fluorescence emission at the emission wavelength of the fluorescent label of a FRET pair indicates that the query probe is bound to an analyte in a FRET method. Binding of the query probe to the target analyte is a “binding event”. In some embodiments of the technology, a binding event has a fluorescence emission having a measured intensity greater or less than a defined threshold.

For example, in some embodiments a binding event has a fluorescence intensity that is above the background fluorescence intensity (e.g., the fluorescence intensity observed in the absence of an analyte). In some embodiments, a binding event has a fluorescence intensity that is at least 1, 2, 3, 4 or more standard deviations above the background fluorescence intensity (e.g., the fluorescence intensity observed in the absence of an analyte). In some embodiments, a binding event has a fluorescence intensity that is at least 2 standard deviations above the background fluorescence intensity (e.g., the fluorescence intensity observed in the absence of an analyte). In some embodiments, a binding event has a fluorescence intensity that is at least 1.5, 2, 3, 4, or 5 times the background fluorescence intensity (e.g., the mean fluorescence intensity observed in the absence of an analyte).

Accordingly, in some embodiments detecting fluorescence at the emission wavelength that has an intensity above the defined threshold (e.g., at least 2 standard deviations greater than background intensity) indicates that a binding event has occurred. Also, in some embodiments detecting fluorescence at the emission wavelength that has an intensity above the defined threshold (e.g., at least 2 standard deviations greater than background intensity) indicates that a binding event has started.

Accordingly, in some embodiments detecting an absence of fluorescence at the emission wavelength of the fluorescent probe that has an intensity above the defined threshold (e.g., at least 2 standard deviations greater than background intensity) indicates that a binding event has ended. The length of time between when the binding event started and when the binding event ended (e.g., the length of time that fluorescence at the emission wavelength of the fluorescent probe having an intensity above the defined threshold (e.g., at least 2 standard deviations greater than background intensity) is detected) is the dwell time of the binding event. A “transition” refers to the binding and dissociation of a query probe to the target analyte (e.g., an on/off event).

Methods according to the technology comprise counting the number of query probe binding events during a defined time interval that is the “acquisition time” or “observation period” (e.g., a time interval that is tens to hundreds to thousands of seconds, e.g., 5, 10, 15, 20, 25, 30, 35, 40, 45, 50, 55, or 60 seconds; e.g., 5, 10, 15, 20, 25, 30, 35, 40, 45, 50, 55, or 60 minutes; e.g., 1, 1.5, 2, 2.5, or 3 hours). In some embodiments, the acquisition time is approximately 1 to 10 seconds to 1 to 10 minutes (e.g., approximately 1 to 100 seconds, e.g., 1, 2, 3, 4, 5, 6, 7, 8, 9, 10, 11, 12, 13, 14, 15, 16, 17, 18, 19, 20, 30, 40, 50, 60, 70, 80, 90, or 100 seconds, e.g., 1 to 100 minutes, e.g., 1, 2, 3, 4, 5, 6, 7, 8, 9, 10, 11, 12, 13, 14, 15, 16, 17, 18, 19, 20, 30, 40, 50, 60, 70, 80, 90, or 100 minutes). In some embodiments, the acquisition time is 0.1-20.0 seconds (e.g., 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, or 20.0 seconds).

Further, the length of time the query probe remains bound to the analyte during a binding event is the “dwell time” of the binding event. The number of binding events detected during the acquisition time and/or the lengths of the dwell times recorded for the binding events is/are characteristic of a query probe binding to an analyte and thus provide an indication that the analyte is present in the sample.

Binding of the query probe to the immobilized analyte and/or dissociation of the query probe from the immobilized analyte is/are monitored (e.g., using a light source to excite a first fluorescent moiety and detecting fluorescence emission from a second fluorescent moiety) and/or recorded during a defined time interval (e.g., during the acquisition time). The number of times the query probe binds to the analyte during the acquisition time and/or the length of time the query probe remains bound to the analyte during each binding event and the length of time the query probe remains unbound to the analyte between each binding event (e.g., the “dwell times” in the bound and unbound states, respectively) are determined, e.g., by the use of a computer and software (e.g., to analyze the data using a hidden Markov model and Poisson statistics).

In some embodiments, control samples are measured (e.g., in absence of analyte). Fluorescence detected in a control sample is “background fluorescence” or “background (fluorescence) intensity” or “baseline”.

In some embodiments, data comprising measurements of fluorescence intensity at the emission wavelength are recorded as a function of time. In some embodiments, the number of binding events and the dwell times of binding events are determined from the data (e.g., by determining the number of times and the lengths of time the fluorescence intensity is above a threshold background fluorescence intensity). In some embodiments, transitions (e.g., binding and dissociation of a query probe) are counted.

In some embodiments, a threshold number of transitions is used to discriminate the presence of an analyte in the sample from background signal, non-target analyte, and/or spurious binding of the query probe. In some embodiments, a number of transitions greater than 2, 3, 4, 5, 6, 7, 8, 9, 10, 11, 12, 13, 14, 15, 16, 17, 18, 19, or 20 recorded during the acquisition time indicates the presence of an analyte in a sample. In some embodiments, a number of transitions greater than 2, 3, 4, 5, 6, 7, 8, 9, 10, 11, 12, 13, 14, 15, 16, 17, 18, 19, or 20 recorded during the acquisition time indicates that the data describing fluorescence emission as a function of time is a candidate signal indicating associated with an analyte in the sample.

In some embodiments, a kinetic parameter is determined. In some embodiments, a kinetic parameter is calculated. For example, in some embodiments, a kinetic parameter is determined and/or calculated from data describing fluorescence emission as a function of time.

In some embodiments, a distribution of the number of transitions for an analyte is determined—e.g., the number of transitions is counted for each analyte observed. In some embodiments, a histogram is produced. In some embodiments, characteristic parameters of the distribution (e.g., of the histogram) are determined, e.g., the mean, median, peak, shape, etc. of the distribution are determined. In some embodiments, data and/or parameters (e.g., fluorescence data (e.g., fluorescence data in the time domain), kinetic data, characteristic parameters of the distribution, etc.) are analyzed by algorithms that recognize patterns and regularities in data, e.g., using artificial intelligence, pattern recognition, machine learning, statistical inference, neural nets, etc. In some embodiments, the analysis comprises use of a frequentist analysis and in some embodiments the analysis comprises use of a bayesian analysis. In some embodiments, pattern recognition systems are trained using known “training” data (e.g., using supervised learning) and in some embodiments algorithms are used to discover previously unknown patterns (e.g., unsupervised learning). See, e.g., Duda, et al. (2001) Pattern classification (2nd edition), Wiley, New York; Bishop (2006) Pattern Recognition and Machine Learning, Springer.

Pattern recognition (e.g., using training sets, supervised learning, unsupervised learning, and analysis of unknown samples) associates identified patterns with analytes such that particular patterns provide a “fingerprint” of particular analytes that find use in detection, quantification, and identification of analytes.

In some embodiments, the distribution produced from an analyte is significantly different than a distribution produced from a non-analyte or the distribution produced in the absence of an analyte. In some embodiments, a mean number of transitions is determined for a plurality of the analyte (e.g., a plurality of individual instances of the same analyte in a sample). In some embodiments, the mean number of transitions observed for a sample comprising an analyte is approximately linearly related as a function of time and has a positive slope (e.g., the mean number of transitions increases approximately linearly as a function of time).

In some embodiments, the data are treated using statistics (e.g., Poisson statistics) to determine the probability of a transition occurring as a function of time. In some particular embodiments, a relatively constant probability of a transition event occurring as a function of time indicates the presence of an analyte. In some embodiments, a correlation coefficient relating event number and elapsed time is calculated from the probability of a transition event occurring as a function of time. In some embodiments, a correlation coefficient relating event number and elapsed time greater than 0.95 when calculated from the probability of a transition event occurring as a function of time indicates the presence of an analyte.

In some embodiments, dwell times of bound query probe (τₒₙ) and unbound query probe (τ_{off}) are used to identify the presence of an analyte in a sample and/or to distinguish a sample comprising an analyte from a sample comprising a non-analyte and/or not comprising an analyte. For example, the τₒₙ for an analyte is greater than the τₒₙ for a non-analyte (e.g., a decoy); and, the τ_{off} for an analyte is smaller than the τ_{off} for a non-analyte (e.g., a decoy). In some embodiments, measuring τₒₙ and τ_{off} for a negative control and for a sample indicates the presence or absence of an analyte in the sample.

In some embodiments, a plurality of τₒₙ and τ_{off} a values is determined for each of a plurality of spots imaged on a solid support, e.g., for a control (e.g., positive and/or negative control) and a sample suspected of comprising an analyte. In some embodiments, a mean τₒₙ and/or τ_{off} is determined for each of a plurality of spots imaged on a solid support, e.g., for a control (e.g., positive and/or negative control) and a sample suspected of comprising an analyte. In some embodiments, a plot of τₒₙ versus τ_{off} (e.g., mean τₒₙ and τ_{off}, time-averaged τₒₙ and τ_{off}, etc.) for all imaged spots indicates the presence or absence of an analyte in the sample.

### Systems

Some embodiments of the technology provide systems for the detection and quantification of an analyte. In some embodiments, systems according to the technology comprise, e.g., a solid support (e.g., a microscope slide, a coverslip, an avidin (e.g., streptavidin)-conjugated microscope slide or coverslip, a solid support comprising a zero mode waveguide array, or the like) and one or more embodiments of compositions described herein (e.g., comprising a query probe moiety and a capture probe moiety and, optionally, comprising a decoy moiety and/or an anchor moiety). Compositions can comprise these various components linked together and/or free in solution as described in the various embodiments herein. Some embodiments of systems comprise an imaging buffer. In some embodiments, an imaging buffer comprises a pH buffer, one or more salts, and water. In some embodiments, an imaging buffer comprises an ionic chaotropic agents. In some embodiments, an imaging buffer comprises a nonionic chaotropic agent. In some embodiments, an imaging buffer comprises guanidine hydrochloride, formamide, or urea. In some embodiments, an imaging buffer comprises approximately 10% formamide (e.g., approximately 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, or 20.0% formamide).

Some embodiments further comprise a fluorescence microscope comprising an illumination configuration to excite bound query probes (e.g., a prism-type total internal reflection fluorescence (TIRF) microscope, an objective-type TIRF microscope, a near-TIRF or HiLo microscope, a confocal laser scanning microscope, a zero-mode waveguide, and/or an illumination configuration capable of parallel monitoring of a large area of the slide or coverslip (>100 μm²) while restricting illumination to a small region of space near the surface). Some embodiments comprise a fluorescence detector, e.g., a detector comprising an intensified charge coupled device (ICCD), an electron-multiplying charge coupled device (EM-CCD), a complementary metal-oxide-semiconductor (CMOS), a photomultiplier tube (PMT), an avalanche photodiode (APD), and/or another detector capable of detecting fluorescence emission from single chromophores. Some embodiments comprise a computer and software encoding instructions for the computer to perform.

Some embodiments comprise optics, such as lenses, mirrors, dichroic mirrors, optical filters, etc., e.g., to detect fluorescence selectively within a specific range of wavelengths or multiple ranges of wavelengths.

Embodiments comprise a source of photons to excite a fluorescent moiety (e.g., a laser or other source of electromagnetic radiation).

In some embodiments, computer-based analysis software is used to translate the raw data generated by the detection assay (e.g., the presence, absence, or amount of one or more analytes) into data of predictive value for a clinician. The clinician can access the predictive data using any suitable means.

For instance, some embodiments comprise a computer system upon which embodiments of the present technology may be implemented. In various embodiments, a computer system includes a bus or other communication mechanism for communicating information and a processor coupled with the bus for processing information. In various embodiments, the computer system includes a memory, which can be a random access memory (RAM) or other dynamic storage device, coupled to the bus, and instructions to be executed by the processor. Memory also can be used for storing temporary variables or other intermediate information during execution of instructions to be executed by the processor. In various embodiments, the computer system can further include a read only memory (ROM) or other static storage device coupled to the bus for storing static information and instructions for the processor. A storage device, such as a magnetic disk or optical disk, can be provided and coupled to the bus for storing information and instructions.

In various embodiments, the computer system is coupled via the bus to a display, such as a cathode ray tube (CRT) or a liquid crystal display (LCD), for displaying information to a computer user. An input device, including alphanumeric and other keys, can be coupled to the bus for communicating information and command selections to the processor. Another type of user input device is a cursor control, such as a mouse, a trackball, or cursor direction keys for communicating direction information and command selections to the processor and for controlling cursor movement on the display.

This input device typically has two degrees of freedom in two axes, a first axis (e.g., x) and a second axis (e.g., y), that allows the device to specify positions in a plane.

A computer system can perform embodiments of the present technology.

Consistent with certain implementations of the present technology, results can be provided by the computer system in response to the processor executing one or more sequences of one or more instructions contained in the memory. Such instructions can be read into the memory from another computer-readable medium, such as a storage device. Execution of the sequences of instructions contained in the memory can cause the processor to perform the methods described herein. Alternatively, hard-wired circuitry can be used in place of or in combination with software instructions to implement the present teachings. Thus, implementations of the present technology are not limited to any specific combination of hardware circuitry and software.

The term “computer-readable medium” as used herein refers to any media that participates in providing instructions to the processor for execution. Such a medium can take many forms, including but not limited to, non-volatile media, volatile media, and transmission media. Examples of non-volatile media can include, but are not limited to, optical or magnetic disks, such as a storage device. Examples of volatile media can include, but are not limited to, dynamic memory. Examples of transmission media can include, but are not limited to, coaxial cables, copper wire, and fiber optics, including the wires that comprise the bus.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

Various forms of computer readable media can be involved in carrying one or more sequences of one or more instructions to the processor for execution. For example, the instructions can initially be carried on the magnetic disk of a remote computer. The remote computer can load the instructions into its dynamic memory and send the instructions over a network connection (e.g., a LAN, a WAN, the internet, a telephone line). A local computer system can receive the data and transmit it to the bus. The bus can carry the data to the memory, from which the processor retrieves and executes the instructions. The instructions received by the memory may optionally be stored on a storage device either before or after execution by the processor.

In accordance with various embodiments, instructions configured to be executed by a processor to perform a method are stored on a computer-readable medium. The computer-readable medium can be a device that stores digital information. For example, a computer-readable medium includes a compact disc read-only memory (CD-ROM) as is known in the art for storing software. The computer-readable medium is accessed by a processor suitable for executing instructions configured to be executed.

In accordance with such a computer system, some embodiments of the technology provided herein further comprise functionalities for collecting, storing, and/or analyzing data (e.g., presence, absence, concentration of a nucleic acid). For example, some embodiments contemplate a system that comprises a processor, a memory, and/or a database for, e.g., storing and executing instructions, analyzing fluorescence, image data, performing calculations using the data, transforming the data, and storing the data. It some embodiments, an algorithm applies a statistical model (e.g., a Poisson model or hidden Markov model) to the data.

Many diagnostics involve determining the presence of, or a nucleotide sequence of, one or more analytes. Thus, in some embodiments, an equation comprising variables representing the presence, absence, concentration, amount, or sequence properties of multiple analytes produces a value that finds use in making a diagnosis or assessing the presence or qualities of a nucleic acid. As such, in some embodiments this value is presented by a device, e.g., by an indicator related to the result (e.g., an LED, an icon on a display, a sound, or the like). In some embodiments, a device stores the value, transmits the value, or uses the value for additional calculations. In some embodiments, an equation comprises variables representing the presence, absence, concentration, amount, or sequence properties of one or more analytes.

Thus, in some embodiments, the present technology provides the further benefit that a clinician, who is not likely to be trained in genetics or molecular biology, need not understand the raw data. The data are presented directly to the clinician in its most useful form. The clinician is then able to utilize the information to optimize the care of a subject. The present invention contemplates any method capable of receiving, processing, and transmitting the information to and from laboratories conducting the assays, information providers, medical personal, and/or subjects. For example, in some embodiments of the present technology, a sample is obtained from a subject and submitted to a profiling service (e.g., a clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center or subjects may collect the sample themselves and directly send it to a profiling center. Where the sample comprises previously determined biological information, the information may be directly sent to the profiling service by the subject (e.g., an information card containing the information may be scanned by a computer and the data transmitted to a computer of the profiling center using electronic communication systems). Once received by the profiling service, the sample is processed and a profile is produced that is specific for the diagnostic or prognostic information desired for the subject. The profile data are then prepared in a format suitable for interpretation by a treating clinician. For example, rather than providing raw expression data, the prepared format may represent a diagnosis or risk assessment for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor. In some embodiments, the information is first analyzed at the point of care or at a regional facility. The raw data are then sent to a central processing facility for further analysis and/or to convert the raw data to information useful for a clinician or patient. The central processing facility provides the advantage of privacy (all data are stored in a central facility with uniform security protocols), speed, and uniformity of data analysis. The central processing facility can then control the fate of the data following treatment of the subject. For example, using an electronic communication system, the central facility can provide data to the clinician, the subject, or researchers. In some embodiments, the subject is able to access the data using the electronic communication system. The subject may chose further intervention or counseling based on the results. In some embodiments, the data are used for research use. For example, the data may be used to further optimize the inclusion or elimination of markers as useful indicators of a particular condition associated with the disease.

### Samples

In some embodiments, analytes are isolated from a biological sample. Analytes can be obtained from any material (e.g., cellular material (live or dead), extracellular material, viral material, environmental samples (e.g., metagenomic samples), synthetic material (e.g., amplicons such as provided by PCR or other amplification technologies)), obtained from an animal, plant, bacterium, archaeon, fungus, or any other organism. Biological samples for use in the present technology include viral particles or preparations thereof.

Analytes can be obtained directly from an organism or from a biological sample obtained from an organism, e.g., from blood, urine, cerebrospinal fluid, seminal fluid, saliva, sputum, stool, hair, sweat, tears, skin, and tissue. Exemplary samples include, but are not limited to, whole blood, lymphatic fluid, serum, plasma, buccal cells, sweat, tears, saliva, sputum, hair, skin, biopsy, cerebrospinal fluid (CSF), amniotic fluid, seminal fluid, vaginal excretions, serous fluid, synovial fluid, pericardial fluid, peritoneal fluid, pleural fluid, transudates, exudates, cystic fluid, bile, urine, gastric fluids, intestinal fluids, fecal samples, and swabs, aspirates (e.g., bone marrow, fine needle, etc.), washes (e.g., oral, nasopharyngeal, bronchial, bronchialalveolar, optic, rectal, intestinal, vaginal, epidermal, etc.), breath condensate, and/or other specimens.

Any tissue or body fluid specimen may be used as a source of analytes for use in the technology, including forensic specimens, archived specimens, preserved specimens, and/or specimens stored for long periods of time, e.g., fresh-frozen, methanol/acetic acid fixed, or formalin-fixed paraffin embedded (FFPE) specimens and samples. Analytes can also be isolated from cultured cells, such as a primary cell culture or a cell line. The cells or tissues from which analytes are obtained can be infected with a virus or other intracellular pathogen. A sample can also be total RNA extracted from a biological specimen, a cDNA library, viral, or genomic DNA. A sample may also be isolated DNA from a non-cellular origin, e.g. amplified/isolated DNA that has been stored in a freezer.

Analytes (e.g., nucleic acid molecules, polypeptides, lipids, metabolites, sugars, etc.) can be obtained, e.g., by extraction from a biological sample, e.g., by a variety of techniques such as those described by Maniatis, et al. (1982) Molecular Cloning: A Laboratory Manual, Cold Spring Harbor, N.Y. (see, e.g., pp. 280-281).

In some embodiments, the technology provides for the size selection of analytes, e.g., to provide a defined size range of molecules including the target analytes.

In some embodiments, single cells (e.g., human, mammalian, vertebrate, eukaryotic, or prokaryotic cells), single organelles (e.g., mitochondria, nuclei, Golgi bodies), subcellular vesicles (e.g., exosomes), and/or subcellular cohesive particles (e.g., membraneless organelles (e.g., intracellular exosome, protein aggregates)) are isolated (e.g., in separate wells, microwells, microfluidic channels, sample chambers, test tubes, microcentrifuge tubes, or other vessel or compartment, or in separate regions of the same compartment). In some embodiments, single cells, organelles, subcellular vesicles, and/or subcellular cohesive particles are isolated by physical extraction (e.g., using suction, fluidic force, or optical trapping) from living or chemically fixed cells, tissues, colonies, or biofluids. In some embodiments, the single cells, organelles, subcellular vesicles, and/or subcellular cohesive particles are isolated from formalin-fixed, paraffin-embedded (FFPE) tissue. In some embodiments, single cells, organelles, subcellular vesicles, and/or subcellular cohesive particles are isolated from suspension by fluidic (e.g., by fluorescence-activated cell sorting, FACS) or microfluidic manipulation, or by segregation into separate wells or regions of a culture dish, microscope slide, or coverslip, or in different regions of a 3D cell culture medium or matrix. In some embodiments, an electric and/or magnetic field is used to effect the separation of a single cell, organelle, subcellular vesicle, and/or subcellular cohesive particle from other biological components. That is, in some embodiments, an electric and/or magnetic field is used to isolate single cells, organelles, subcellular vesicles, and/or subcellular cohesive particles.

In some embodiments, single cells (e.g., human, mammalian, vertebrate, eukaryotic, or prokaryotic cells), single organelles (e.g., mitochondria, nuclei, Golgi bodies), subcellular vesicles (e.g., exosomes), and/or subcellular cohesive particles (e.g., membraneless organelles (e.g., intracellular exosome, protein aggregates)) are treated (e.g., with cell lysis buffers; detergents such as SDS, Triton X-100, or deoxycholate; and/or enzymes such as proteases, lipases, or nucleases) to release an analyte from each single cell, organelle, subcellular vesicle, and/or subcellular cohesive particle. In some embodiments, single cells, organelles, subcellular vesicles, and/or subcellular cohesive particles are treated in situ on or near a solid support to liberate the analyte for capture on a nearby region of the solid support.

In some embodiments, the analyte is detected by a single molecule detection method (e.g., SiMREPS).

Uses Various embodiments relate to the detection of a wide range of analytes. For example, in some embodiments the technology finds use in detecting a nucleic acid (e.g., a DNA or RNA). In some embodiments, the technology finds use in detecting a nucleic acid comprising a particular target sequence. In some embodiments, the technology finds use in detecting a nucleic acid comprising a particular mutation (e.g., a single nucleotide polymorphism, an insertion, a deletion, a missense mutation, a nonsense mutation, a genetic rearrangement, a gene fusion, etc.). In some embodiments, the technology finds use in detection a polypeptide (e.g., a protein, a peptide). In some embodiments, the technology finds use in detecting a polypeptide encoded by a nucleic acid comprising a mutation (e.g., a polypeptide comprising a substitution, a truncated polypeptide, a mutant or variant polypeptide).

In some embodiments, the technology finds use in detecting post-translational modifications to polypeptides (e.g., phosphorylation, methylation, acetylation, glycosylation (e.g., O-linked glycosylation, N-linked glycosylation, ubiquitination, attachment of a functional group (e.g., myristoylation, palmitoylation, isoprenylation, prenylation, farnesylation, geranylation, geranylgeranylation, glypiation, glycosylphosphatidylinositol (GPI) anchor formation), hydroxylation, biotinylation, pegylation, oxidation, SUMOylation, disulfide bridge formation, disulfide bridge cleavage, proteolytic cleavage, amidation, sulfation, pyrrolidone carboxylic acid formation. In some embodiments, the technology finds use in the detection of the loss of these features, e.g., dephosporylation, demethylation, deacetylation, deglycosylation, deamidation, dehydroxylation, deubiquitination, etc. In some embodiments, the technology finds use in detecting epigenetic modifications to DNA or RNA (e.g., methylation (e.g., methylation of CpG sites), hydroxymethylation). In some embodiments, the technology finds use in detecting the loss of these features, e.g., demethylation of DNA or RNA, etc. In some embodiments, the technology finds use in detecting alterations in chromatin structure, nucleosome structure, histone modification, etc., and in detecting damage to nucleic acids.

In some embodiments, the technology finds use as a molecular diagnostic assay, e.g., to assay samples having small specimen volumes (e.g., a droplet of blood, e.g., for mail-in service). In some embodiments, the technology provides for the early detection of cancer or infectious disease using sensitive detection of very low-abundance analyte biomarkers. In some embodiments, the technology finds use in molecular diagnostics to assay epigenetic modifications of protein biomarkers (e.g., post-translational modifications).

In some embodiments, the technology finds use in characterizing multimolecular complexes (e.g., characterizing one or more components of a multimolecular complex), e.g., a multiprotein complex, a nucleic acid/protein complex, a molecular machine, an organelle (e.g., a cell-free mitochondrion, e.g., in plasma), cell, virus particle, organism, tissue, or any macromolecular structure or entity that can be captured and is amenable to analysis by the technology described herein. For example, in some embodiments a multimolecular complex is isolated and the technology finds use in characterizing, identifying, quantifying, and/or detecting one or more molecules (analytes) associated with the multimolecular complex. In some embodiments an extracellular vesicle is isolated and the technology finds use in characterizing, identifying, quantifying, and/or detecting one or more molecules (analytes) associated with the vesicle. In some embodiments, the technology finds use in characterizing, identifying, quantifying, and/or detecting a protein (e.g., a surface protein) and/or an analytes present inside the vesicle, e.g., a protein, nucleic acid, or other analyte described herein. In some embodiments, the vesicle is fixed and permeabilized prior to analysis.

Although the disclosure herein refers to certain illustrated embodiments, it is to be understood that these embodiments are presented by way of example and not by way of limitation.

### EXAMPLES

### Example 1

During the development of embodiments of the technology described herein, experiments were conducted to test an embodiment of a biosensor comprising three nucleic acid strands in addition to the target nucleic acid (e.g., miR-141).

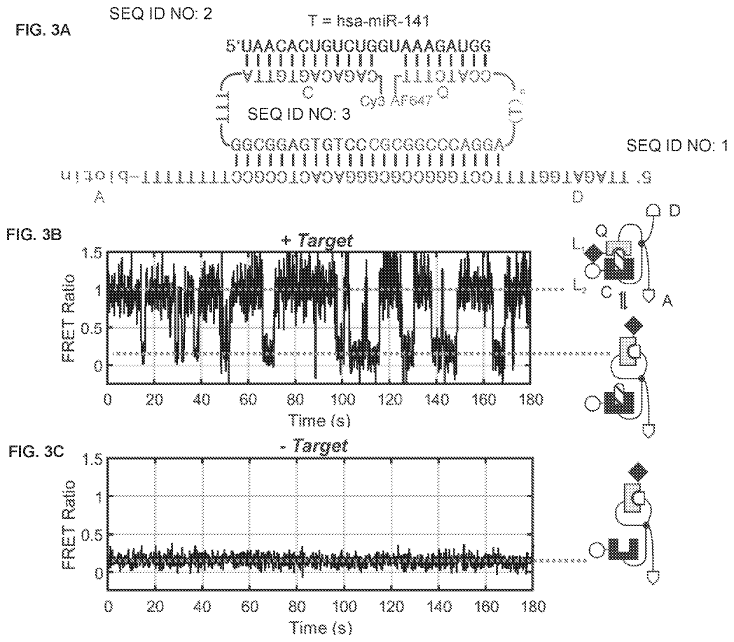

FIG. 3a shows an embodiment of a biosensor design comprising three nucleic acid strands in addition to the target nucleic acid (T), the microRNA hsa-miR-141. The capture (C) and query (Q) probes are long and short nucleic acid sequences that bind to adjacent complementary regions on T, resulting in juxtaposition of the labels Alexa Fluor 647 (AF647, L1) and Cy3 (L2) when both C and Q are bound to T.

FIG. 3b and FIG. 3c show data collected from an experimental demonstration of an embodiment of the biosensor. The juxtaposition of Cy3 and AF647 permits Forster resonance energy transfer (FRET) between the two dyes, and as a result nearly 100% of fluorescent signal from the complex is emitted from AF647. When Q is not bound to T, Q instead binds to decoy sequence D, resulting in low FRET efficiency and nearly 100% of fluorescent from the complex is emitted from Cy3. The capture (C) sequence incorporates locked nucleic acid (LNA) modifications for greater stability of target binding by the C probe. FIG. 3b shows FRET measurement of a single biosensor molecule with hsa-miR-141 bound. Repeated transitions between a high-FRET state (mostly Cy5 emission, red) and a low-FRET state (mostly Cy3 emission, blue) are seen at t=19 s and t=47 seconds, permitting kinetic characterization of the interaction with T and thus providing a clear signature of the presence of T. FIG. 3c shows a FRET measurement in the absence of T, showing only the low-FRET state and thus indicating the absence of T.

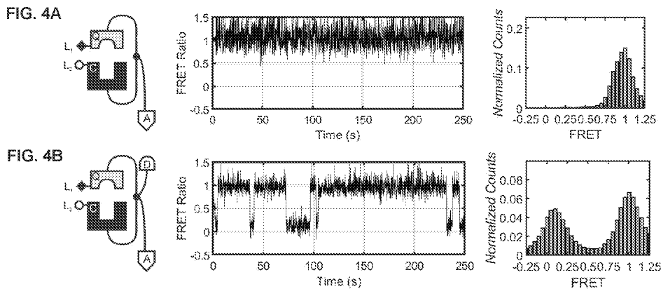

FIG. 4a and FIG. 4b show additional data collected during experiments conducted to test the influence of the decoy on the signal resulting from an intramolecular sensor described above. In particular, experiments were conducted to test an embodiment of the biosensor as described above (e.g., comprising three nucleic acid strands in addition to the target nucleic acid, miR-141) in the presence and absence of the decoy (D).

Plots on the left of FIG. 4a and FIG. 4b show representative single-molecule FRET trajectories from individual biosensors; plots on the right of FIG. 4a and FIG. 4b are histograms of FRET Ratios observed in 36 biosensors (from each condition) exhibiting at least one data point of non-zero FRET efficiency.

FIG. 4a depicts data resulting from single-molecule FRET measurements of a biosensor in which a decoy (D) is absent, resulting in a stable FRET efficiency close to 1.

FIG. 4b depicts data from single-molecule FRET measurements of a biosensor in which a 7-nucleotide decoy sequence is present, resulting in dynamic transitions between FRET efficiencies of approximately 1 and 0.1. Thus, the presence of the decoy produces observable FRET transitions that were not observed for the biosensor without the decoy.

### Example 2

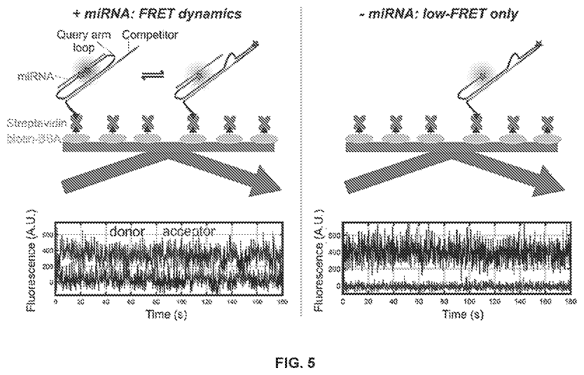

During the development of embodiments of the technology described herein, experiments were conducted to improve the imaging time of the technology to approximately 10 seconds per target nucleic acid molecule. In particular, experiments were conducted to collect data characterizing FRET states for decoys (D) comprising spacers of different lengths and/or query probes comprising spacers (query arm loop) of different lengths in detecting a miRNA target. In these experiments, the target analyte (T), query probe (Q), and decoy (D, “competitor”) were nucleic acids. See, e.g., FIG. 5.

Data were collected to characterize the time-dependent lifetime of the query-target complex (QT), which was detectable by a high FRET signal (the “high-FRET state”) and the time-dependent lifetime of the query-decoy (QD) or free query probe state, which was detectable by a low FRET signal (the “low-FRET state”), and the switching of the system between the high-FRET and low-FRET states. See, e.g., FIG. 5. Without being bound by theory, it was contemplated that the length of the linker comprising the decoy would modulate the dwell time in the low-FRET state and the length of the query arm loop would modulate the flexibility of the query arm and would accordingly influence both the kinetics and thermodynamics of switching between the high-FRET and low-FRET states.

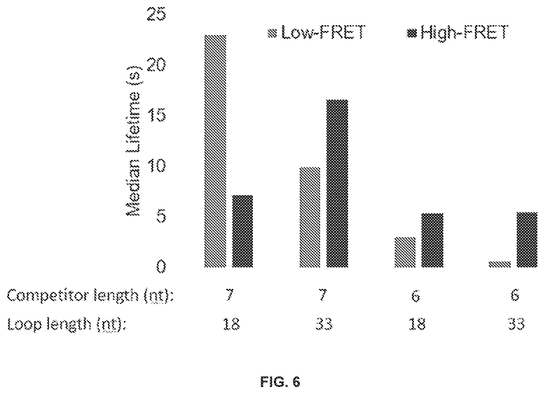

The data collected during these experiments indicated that a decoy comprising a shorter spacer of 6 nucleotides yielded faster switching kinetics and an increased bias towards the high-FRET state (see FIG. 6). However, the data surprisingly indicated that a query probe comprising a longer spacer resulted in a stronger bias towards the high-FRET state (FIG. 6). Without being bound by theory, it was contemplated that altering the equilibrium length of the query arm increased the thermodynamic stability of query probe binding to the target miRNA. The data indicated that the best design among the variations designs tested provided rapid switching kinetics and equal sampling of high-FRET and low-FRET states—the best design used a decoy comprising 6 nt and a query probe comprising an 18-nt query arm loop. This system exhibited approximately 14 transitions per minute, which provides an embodiment of technology that detects an unamplified miRNA target with only 1-2 minutes (e.g., 50, 55, 60, 65, 70, 75, 80, 85, 90, 95, 100, 105, 110, 115, 120, 125, or 130 seconds) of acquisition time.

### Example 3

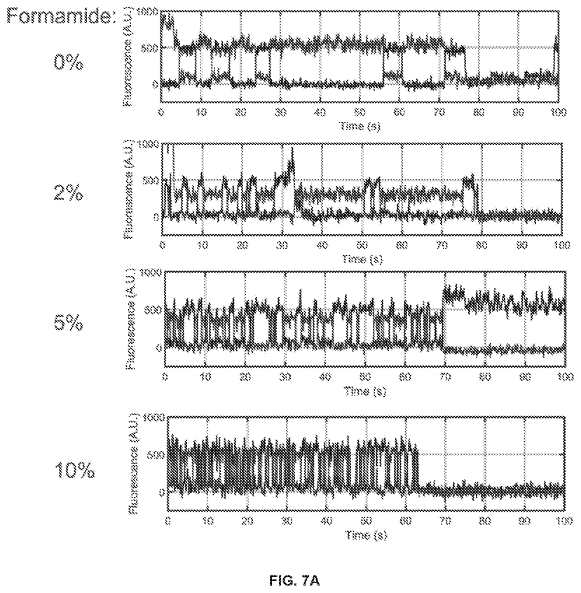

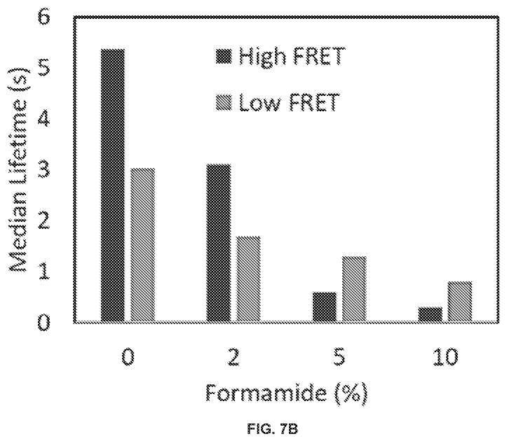

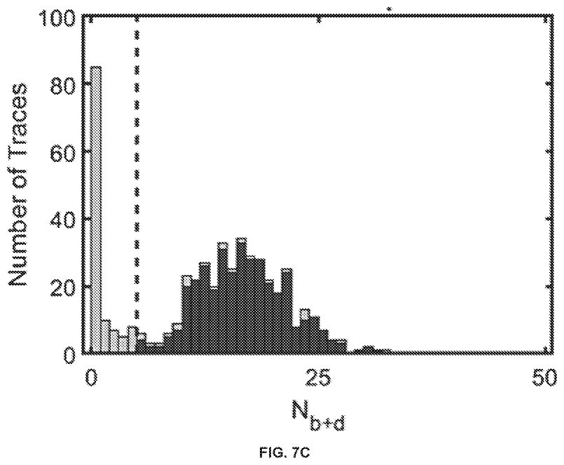

Based on the data collected in the experiments described above, it was contemplated that further decreases in the imaging time would be provided by reducing the number of base pairs formed between the query probe and the target miRNA. However, this could also risk reducing the specificity of the query probe for the target miRNA and the query probe may bind to other non-target nucleic acids. Therefore, during the development of embodiments of the technology provided herein experiments were conducted to test using different imaging buffer conditions to accelerate the switching kinetics without reducing the number of complementary base pairs between the query probe and target miRNA. The data indicated that reducing the ionic strength from approximately 600 mM to 150 mM did not provide a significant acceleration of switching kinetics. However, the data indicated that adding 1-10% formamide into the imaging buffer provided increased switching kinetics between the high-FRET and low-FRET states detecting a target miRNA (FIG. 7A and FIG. 7B). Importantly, the data indicated that the technology provided a median dwell time of 1 second for embodiments comprising 10% formamide (FIG. 7A and FIG. 7B). Analysis of only the first 10 seconds of data collected using an imaging buffer comprising 10% formamide (e.g., approximately 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, or 20.0% formamide) revealed that most molecules exhibit approximately 10-25 FRET transitions within this time interval (FIG. 7C). Accordingly, in some embodiments, the technology provides measurements of target presence, amount, and/or concentration that are performed in only approximately 10 seconds (e.g., approximately 1.00, 1.10, 1.20, 1.30, 1.40, 1.50, 1.60, 1.70, 1.80, 1.90, 2.00, 2.10, 2.20, 2.30, 2.40, 2.50, 2.60, 2.70, 2.80, 2.90, 3.00, 3.10, 3.20, 3.30, 3.40, 3.50, 3.60, 3.70, 3.80, 3.90, 4.00, 4.10, 4.20, 4.30, 4.40, 4.50, 4.60, 4.70, 4.80, 4.90, 5.00, 5.10, 5.20, 5.30, 5.40, 5.50, 5.60, 5.70, 5.80, 5.90, 6.00, 6.10, 6.20, 6.30, 6.40, 6.50, 6.60, 6.70, 6.80, 6.90, 7.00, 7.10, 7.20, 7.30, 7.40, 7.50, 7.60, 7.70, 7.80, 7.90, 8.00, 8.10, 8.20, 8.30, 8.40, 8.50, 8.60, 8.70, 8.80, 8.90, 9.00, 9.10, 9.20, 9.30, 9.40, 9.50, 9.60, 9.70, 9.80, 9.90, 10.00, 10.10, 10.20, 10.30, 10.40, 10.50, 10.60, 10.70, 10.80, 10.90, 11.00, 11.10, 11.20, 11.30, 11.40, 11.50, 11.60, 11.70, 11.80, 11.90, 12.00, 12.10, 12.20, 12.30, 12.40, 12.50, 12.60, 12.70, 12.80, 12.90, 13.00, 13.10, 13.20, 13.30, 13.40, 13.50, 13.60, 13.70, 13.80, 13.90, 14.00, 14.10, 14.20, 14.30, 14.40, 14.50, 14.60, 14.70, 14.80, 14.90, 15.00, 15.10, 15.20, 15.30, 15.40, 15.50, 15.60, 15.70, 15.80, 15.90, 16.00, 16.10, 16.20, 16.30, 16.40, 16.50, 16.60, 16.70, 16.80, 16.90, 17.00, 17.10, 17.20, 17.30, 17.40, 17.50, 17.60, 17.70, 17.80, 17.90, 18.00, 18.10, 18.20, 18.30, 18.40, 18.50, 18.60, 18.70, 18.80, 18.90, 19.00, 19.10, 19.20, 19.30, 19.40, 19.50, 19.60, 19.70, 19.80, 19.90, or 20.00 seconds) per target analyte (e.g., an miRNA target).

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

We claim:

1. A biosensor comprising a capture probe moiety and a query probe moiety, wherein:
a) said capture probe moiety comprises a first label and said query probe moiety comprises a second label; and
b) said capture probe binds stably to a target analyte and said query probe moiety binds transiently to said target analyte.

2. The biosensor of claim 1 wherein said first label and said second label are a FRET pair.

3. The biosensor of claim 1 wherein said first label and said second label or a fluor-quencher pair.

4. The biosensor of claim 1 wherein said capture probe moiety binds to said target analyte to form a complex comprising said capture probe moiety and said target analyte, said complex having a melting temperature more than 10° C. above the measurement temperature of the assay.

5. The biosensor of claim 1 wherein said capture probe moiety binds to said target analyte to form a complex comprising said capture probe moiety and said target analyte, said complex having an average lifetime that is more than 5 times as long as the observation period.

6. The biosensor of claim 1 wherein said query probe moiety transiently binds to said target analyte to form a complex comprising said capture query probe moiety and said target analyte, said complex having a melting temperature within 10° C. of the measurement temperature of the assay.

7. The biosensor of claim 1 wherein said query probe moiety transiently binds to said target analyte to form a complex comprising said capture query probe moiety and said target analyte, said complex having an average lifetime that no more than ⅕ as long as the observation period.

8. The biosensor of claim 1 wherein said capture probe moiety and said query probe moiety are covalently linked.

9. The biosensor of claim 1 further comprising a covalently attached anchor moiety.

10. The biosensor of claim 1 further comprising a decoy moiety.

11. The biosensor of claim 10 wherein said decoy moiety transiently binds to said target analyte to form a complex comprising said capture query probe moiety and said target analyte, said complex having a melting temperature within 10° C. of the measurement temperature of the assay.

12. The biosensor of claim 10 wherein said decoy moiety transiently binds to said target analyte to form a complex comprising said capture query probe moiety and said target analyte, said complex having an average lifetime that is no more than ⅕ as long as the observation period.

13. The biosensor of claim 10 wherein said decoy moiety comprises a label or quencher.

14. The biosensor of claim 1 wherein said capture probe moiety comprises an antibody.

15. The biosensor of claim 1 wherein said query probe moiety comprises an antibody.

16. The biosensor of claim 1 wherein said capture probe moiety comprises a nucleic acid.

17. The biosensor of claim 1 wherein said query probe moiety comprises a nucleic acid.

18. A method for detecting an analyte, said method comprising:
a) contacting an analyte with a biosensor comprising a capture probe moiety and a query probe moiety, wherein said capture probe moiety comprises a first label and said query probe moiety comprises a second label; and
b) recording a fluorescence signal indicating the association and dissociation of the query probe with the analyte as a function of time to produce fluorescence transition data.

19. The method of claim 18 comprising forming a thermodynamically stable complex between said capture probe and said analyte.

20. The method of claim 18 comprising repeatedly forming a transient complex between said query probe and said analyte.

21. The method of claim 18 further comprising counting fluorescence transitions in said fluorescence transition data.

22. The method of claim 18 wherein said biosensor comprises an anchor moiety and said method comprising immobilizing said biosensor to a surface.

23. The method of claim 18 further comprising providing or obtaining a sample.

24. The method of claim 18 further comprising providing a decoy moiety.

25. The method of claim 18 further comprising contacting said query probe with a decoy moiety.

26. The method of claim 18 further comprising providing an excitation wavelength.

27. The method of claim 18 further comprising detecting an emission wavelength.

28. The method of claim 18 further comprising recording an emission wavelength as a function of time.

29. The method of claim 18 further comprising recording a time-dependent signal of an emission wavelength.

30. The method of claim 18 further comprising monitoring fluorescence at a discrete location on a solid support.

31. The method of claim 18 further comprising counting binding events.

32. The method of claim 18 further comprising counting fluorescent transition events.

33. The method of claim 18 further comprising measuring the dwell time of binding events.

34. The method of claim 18 further identifying a candidate signal from a time-dependent fluorescence signal comprising at least 5 transitions during the acquisition time.

35. The method of claim 18 further comprising calculating a kinetic parameter, distribution of the number of transitions, and/or a parameter characterizing a distribution.

36. The method of claim 18 further comprising using pattern recognition to process said fluorescence transition data.

37. The method of claim 18 further comprising statistically analyzing said fluorescence transition data.

38. The method of claim 18 further comprising detecting the analyte using said fluorescence transition data.

39. A system for detecting an analyte, said system comprising:
a) a biosensor comprising a capture probe moiety and a query probe moiety, wherein said capture probe moiety comprises a first label and said query probe moiety comprises a second label; and
b) a component configured to record a fluorescence signal indicating the association and dissociation of the query probe with the analyte as a function of time.

40. The system of claim 39 further comprising a solid support.

41. The system of claim 39 further comprising a decoy moiety.

42. The system of claim 39 further comprising a fluorescence microscope.

43. The system of claim 39 further comprising an excitation source.

44. The system of claim 39 further comprising an emission detector.

45. The system of claim 39 further comprising a computer to record and/or analyze fluorescence transition data.

46. The system of claim 39 further comprising an analyte.

47. The system of claim 39 wherein said capture probe moiety comprises an antibody.

48. The system of claim 39 wherein said capture probe moiety comprises a nucleic acid.

49. The system of claim 39 wherein said query probe moiety comprises an antibody.

50. The system of claim 39 wherein said query probe moiety comprises a nucleic acid.

51. The system of claim 41 wherein said decoy moiety comprises an antibody.

52. The system of claim 41 wherein said decoy moiety comprises a nucleic acid.

53. Use of a biosensor of claim 1 to detect an analyte.

54. Use of a method of claim 18 to detect an analyte.

55. Use of a system of claim 39 to detect an analyte.
